(19) United States
(12) Patent Application Publication
(10) Pub. No.: US 2019/0117657 A1
(43) Pub. Date: Apr. 25, 2019

(54) METHOD OF INHIBITING HIGH FAT DIET-RELATED CONDITIONS

(71) Applicants: South Australian Health and Medical Research Institute Limited, Adelaide, South Australia (AU); Ocean University of China, Qingdao (CN)

(72) Inventors: Christopher G. Proud, Unley Park, South Australia (AU); Claire E.J. Moore, Leicester (GB); Rebecca L. Stead, Surrey (GB); Jiang Tao, Qingdao (CN)

(21) Appl. No.: 16/089,637

(22) PCT Filed: Mar. 31, 2017
(86) PCT No.: PCT/AU2017/000077

§ 371 Date: Sep. 28, 2018

(30) Foreign Application Priority Data
Mar. 31, 2016 (AU) 2016901192

(51) Int. Cl. A61K 31/519; A61P 3/10; A61P 5/48

(52) U.S. Cl. CPC A61K 31/519; A61P 5/48; A61P 3/10

(57) ABSTRACT

A method of treating a pre-diabetic subject is disclosed wherein the subject is characterised by having a fasting plasma glucose level from 5.5 mmol/l to 6.9 mmol/l. The method comprises administering to the subject a therapeutically effective amount of at least one mitogen-activated protein kinase-interacting kinase (MNK) inhibitor, wherein said MNK inhibitor reduces the biological activity of MNK1 and/or MNK2. The method may prevent and/or delay progression of pre-diabetes to type 2 diabetes. The method may also prevent progression of pre-diabetes at the impaired fasting glucose (IFG) stage to the impaired glucose tolerance (IGT) stage.

### TECHNICAL FIELD

The present disclosure relates to inhibiting the effects of a high fat diet, particularly pre-diabetes. In a particular form, the present disclosure relates to inhibiting the expression or function of MAP kinase-interacting kinases.

### PRIORITY DOCUMENT

The present application claims priority from Australian Provisional Patent Application No 2016901192 titled “Method of inhibiting high fat diet-related conditions” filed on 31 Mar. 2016, the content of which is hereby incorporated by reference in its entirety.

### BACKGROUND

The consequences of over-consumption of food, in particular saturated fat, pose a major and rapidly-increasing health problem worldwide, resulting in obesity and associated conditions such as adipose tissue inflammation, insulin resistance and type 2 diabetes. Nearly 1 in 4 adults in the western world are either diabetic or pre-diabetic, and incidence is rising due to the increased prevalence of obesity. Pre-diabetes is a condition associated with blood glucose levels that are abnormally high, but not sufficiently high enough to diagnose the person as diabetic. Pre-diabetics are frequently overweight, and the condition is generally associated with an unhealthy diet, such as a high fat diet. Accordingly, the management of pre-diabetes frequently involves the recommendation of weight loss through a low fat diet. However, many patients with pre-diabetes are either unwilling or unable to adhere to the required life style changes to achieve and sustain the recommended weight loss to treat their condition; or alternatively, existing therapies may have failed. There is accordingly a need for alternative treatments and/or management of pre-diabetes.

The mitogen-activated protein (MAP) kinase-interacting kinases (MNKs) are a family of serine/threonine kinases, which are downstream effectors of MAPK signalling, and have been implicated in oncogenic transformation and progression. Murine MNK1 and MNK2 are encoded by the Mknk1 and Mknk2 genes respectively. The corresponding proteins (MNK1 and MNK2) interact with MAP kinases (eg extracellular signal-regulated kinases (ERK)) and, particularly in the case of MNK1, p38 MAP kinase. The MNK proteins are phosphorylated by the MAP kinases, resulting in stimulation of the MNK activities (Waskiewicz et al., 1997). The best-known substrate for the MNK proteins is eukaryotic translation initiation factor 4E (eIF4E), a key component of the protein synthesis machinery (Waskiewicz et al., 1997; Scheper et al., 2001), although a few additional substrates have SUBSTITUTE SPECIFICATION (CLEAN) Attorney Docket No.: 10945-003US1 been described (reviewed in Buxade et al., 2008). Since MNKs are the only kinases acting on elF4E, the biological activity of MNK proteins can be measured by measuring levels of phosphorylated eIF4E (P-eIF4E).

Although very similar in terms of their primary sequences, MNK1 and MNK2 differ in a number of key respects. For example, murine MNK1 (equivalent of human MNK1a) is mainly cytoplasmic while murine MNK2 (similar to human MNK2a) is found in the nucleus and the cytoplasm. MNK1 is strongly activated following stimulation of the ERK or p38 MAP kinase pathway (Scheper et al., 2001; Waskiewicz et al., (1999); Wang et al., 1999), whereas MNK2 shows high basal activity that is only slightly further stimulated by these pathways. MNK1 and MNK2 mRNA are known to be expressed in liver, skeletal muscle and heart; however, the expression patterns of MNK1 and MNK2 in different mouse tissues varies, suggesting MNK1 and MNK2 play distinct roles. Disruption of one or both of the MKNK1 and MKNK2 genes in knockout mice had no reported adverse effects, with double MNK1/2 knockout mice being viable and fertile with no reported abnormalities (Ueda et al., 2004).

The present inventors have realised that MNK activity may be associated with the effects of a high fat diet including obesity, adipogenesis and lipogenesis, and associated conditions such as adipose tissue inflammation, insulin resistance, glucose intolerance, pre-diabetes and type 2 diabetes. As described herein, inhibiting MNK expression or biological activity may inhibit the effects of a high fat diet and provide a new approach to treating and/or managing high fat diet-related conditions such as pre-diabetes.

### SUMMARY

According to a first aspect of the present disclosure, there is provided a method of treating a subject with impaired fasting glucose pre-diabetes to prevent and/or delay progression of the pre-diabetes to type 2 diabetes, said subject characterised by having a fasting plasma glucose level from 5.5 mmol/l to 6.9 mmol/l, wherein said method comprises administering to the subject a therapeutically effective amount of at least one mitogen-activated protein kinase-interacting kinase (MNK) inhibitor, wherein said MNK inhibitor reduces the biological activity of MNK2 and, optionally, MNK1.

In an embodiment, the MNK inhibitor is a small organic molecule, a peptide inhibitor, an inhibitory antibody or fragment thereof, interfering nucleotide molecule, or an aptamer.

### BRIEF DESCRIPTION OF FIGURES

FIG. 1 provides analysis of MKNK1 and MKNK2 mRNA expression, with (A) relative expression of MKNK1 (MNK1) and MKNK2 (MNK2) determined by qPCR in liver and gonadal adipose tissue (n=8), data are mean±SEM (2-tailed, unpaired Student's t test comparing MNK1 with MNK2 for each tissue); (B) immunoblot analysis of MNK1 protein levels in liver, adipose tissue, cardiac and skeletal muscle, representative of 4 independent experiments; (C) mRNA expression of Pparγ, Cebpa, Srebp1c, Glut4 and Cd36 in differentiating 3T3-L1 adipocytes (n=3), data are mean±SEM; (D) Mknk1 (MNK1) and Mknk2 (MNK2) mRNA expression in differentiating 3T3L1 adipocytes (n=3), data are mean±SEM (one-way ANOVA with Tukey's post-test) ** P<0.01, *** P<0.001, ***P<0.0001; (E) graphical results showing expression of Mknk1, Mknk2 and Cebpβ mRNA expression over time (following addition of the differentiation cocktail) in the 3T3-L1 model of adipocyte differentiation;

FIG. 2 provides responses of MNK1 knockout (KO) and MNK2 KO mice to high fat feeding, with (A) bodyweight of wild type (WT), MNK1-KO and MNK2-KO after 20 weeks on either a chow or high fat diet (HFD), (n=6-9), data are mean±SEM (2-tailed, unpaired Student's t test) * P<0.05, ** P<0.01, ****P<0.0001; (B) gonadal adipose tissue weight expressed as a percentage of bodyweight (n=6-9), data are mean±SEM (2-tailed, unpaired Student's t test) * P<0.05, ** P<0.01, *** P<0.001, ****P<0.0001; (C) representative images showing gonadal fat deposits from WT, MNK1-KO and MNK2-KO mice after 20 weeks HFD; (D) fasting blood glucose of WT, MNK1-KO and MNK2-KO after 20 weeks on either a chow or HFD, n=6-9, data are mean±SEM (2-tailed, unpaired Student's t test) * P<0.05, *** P<0.001, (E) fasting plasma insulin of WT, MNK1-KO and MNK2-KO after 20 weeks on either a chow or HFD (n=3-5). Data are mean±SEM; (F) homeostasis model assessment of insulin resistance (HOMA-IR) as a “read-out” of insulin resistance;

FIG. 3 provides effects of knocking out or inhibiting MNKs on adipocytes, with (A) the average sizes of fat cells in WT and MNK2-KO mice analysed and quantified by image J software (WT chow, n=3, WT HFD n=3, MNK2-KO chow n=3, MNK2-KO HFD n=3), data are mean±SEM (2-tailed, unpaired Student's t test), **P<0.01; (B) the average number of adipocytes in two fields of view for the following conditions (WT chow n=3, WT HFD n=3, MNK2-KO chow n=3, MNK2-KO HFD n=3), data are mean±SEM (2-tailed, unpaired Student's t test) *P<0.05; (C) (i) immunoblot analysis of lysates of undifferentiated 3T3-L1 cells treated with the indicated concentrations of CGP57380 for 72 h, representative of three independent experiments; (ii) quantification of the data shown in (i), data are mean±SEM (one-way ANOVA with Tukey's post-test) ****P<0.0001; (D) Oil Red O staining of 3T3-L1 cells differentiated for 9 days in the absence or presence of 20 μM CGP57380, representative microscopic fields of view are shown, (n=3); data are mean+SEM (2-tailed, unpaired Student's t test); (E) mRNA expression of Pparγ, Cbpa, Srebp1c, Glut4 and Cd36 in 3T3-L1 adipocytes subjected to the differentiation protocol in the presence or absence of 20 μM CGP57380 (n=3); data are mean+SEM (one-way ANOVA with Tukey's post-test);

FIG. 4 provides graphical results of metabolic studies of HFD-fed MNK1-KO or MNK2-KO mice, with (A) glucose tolerance test after 15 weeks on either the chow or HFD (n=6-21), data are mean+SEM; (B) area under the curve calculations (AUC) (n=6-21), data are mean+SEM (two-way ANOVA with Tukey's post-test); (C) insulin resistance test after 20 weeks on either chow or HFD the mice (n=3-4), data are mean+SEM (2-tailed, unpaired Student's t test) * P<0.05, ** P<0.01;

FIG. 5 provides immunoblot analysis of total protein lysates of tissues of chow fed or HFD fed WT, MNK1-KO and MNK2-KO mice showing (A) analysis of P-eIF4E and eIF4E levels in gonadal adipose tissue, representative of 3 independent experiments; (B) analysis of levels of PKB, phosphorylated PKB and actin in gonadal adipose tissue following insulin resistance testing, (n=3); (C) immunoblot analysis of levels of PKB, phosphorylated PKB and tubulin in skeletal muscle from mice treated (where indicated) with an intraperitoneal injection of 0.75 U/kg insulin and sacrificed 30 min later, and tissues were collected for immunoblot analysis using the indicated antibodies; (D) upper panel, analysis of GLUT 4 levels; lower panel, quantitation of data in (D, upper panel) from three mice in each case, expressed as GLUT4 normalised to actin, data are mean+SEM (2-tailed, unpaired Student's t test) * P<0.05; (E) analysis of P-eIF4E and eIF4E levels in skeletal muscle (n=3); (F) quantitation of data for GLUT4 protein levels in skeletal muscle (normalised to eIF4E) from three mice in each case;

FIG. 6 provides qPCR analysis of total RNA isolated from gonadal adipose tissue from chow fed and HFD fed WT, MNK1-KO and MNK2-KO mice for macrophage markers in adipose tissue from WT and MNK-KO mice, showing relative expression of mRNA for (A) general macrophage marker Cd68, (B) general macrophage marker F4/80, (C) M1 polarised macrophage marker Tnfa, (D) M1 polarised macrophage marker Cd11c, (E) M1 polarised macrophage marker MhcII (n=3-4), data are mean+SEM (2-tailed, unpaired Student's t test) * P<0.05, ** P<0.01;

FIG. 7 provides analysis of plasma levels of (A) IL-5 and (B) IL-10 from chow fed and HFD fed WT, MNK1-KO and MNK2-KO mice measured by ELISA (n=6-9), data are mean+SEM (2-tailed, unpaired Student's t test) * P<0.05; and qPCR analysis of total RNA isolated from bone marrow-derived macrophage (BMDMs) isolated from WT or MNK2-KO mice cultured for 24 h in the presence or absence of IL-4 to polarise the BMDMs towards an M2 phenotype for mRNA expression levels of M2 markers (C) 1l-10, (D) Cd206, (E) Pparγ and (F) Stat6 (n=6), data are mean±SEM (2-tailed, unpaired Student's t test) * P<0.05, ** P<0.01, *** P<0.001, ****P<0.0001;

FIG. 8 provides qPCR analysis of total RNA isolated from liver tissue of chow fed and HFD fed WT and MNK2-KO mice for the relative expression of mRNA for (A) Nrf2 and (B) its downstream target heme oxygenase 1 (Ho-1), (n=4), data are mean±SEM (2-tailed, unpaired Student's t test) * P<0.05; and qPCR analysis of the relative mRNA expression of genes in the liver from chow-fed and HFD-fed WT and MNK2-KO mice involved in de nova lipogenesis (C) Srebp1c, (D) Fas, (E) Acc1 and (F) β-oxidation (Cpt1a), (n=4), data are mean±SEM (2-tailed, unpaired Student's t test) * P<0.05, ** P<0.01;

FIG. 9 provides an immunoblot analysis of lysates from C2C12 skeletal muscle cells that had been treated for 16 hours with 4 mM of the fatty acid palmitate prior to stimulation with 100 nM insulin (for 10 and 60 minutes) and, where indicated, with 3 μM of the MNK inhibitor, MNK-I1. Palmitate induces insulin resistance in these cells, as shown by the impaired ability of insulin to increase the phosphorylation of PKB at the key regulatory site, Thr308, in cells that have been pre-treated with palmitate. The levels of the marker P-308-PKB showed that the MNK inhibitor is able to restore insulin signalling following exposure of cells to palmitate;

FIG. 10 provides graphical results showing the response on MNK1-KO, MNK2-KO and MNK1+MNK2 double KO (DKO) animals to calorie-overload (ie by feeding on an energy-rich high fat diet (HFD) from 4 weeks to 15 weeks of age); shown are data for weight gain of HFD minus weight gain of chow-fed animals. 6-16/group. *, p<0.05 Two-tailed, unpaired t test;

FIG. 11 provides the results of glucose tolerance tests (GTTs), shown as plasma glucose concentration, in MNK1-KO and DKO mice compared to WT mice fed on chow (CD) or HFD;

FIG. 12 provides a bar graph showing MNK2 expression over time (0 to 24 hr) in 3T3-L1 fibroblasts treated with “differentiation cocktail” for the indicated times; cells were lysed and RT-qPCR analyses performed for expression of the Mnk2 mRNA. Data are expressed relative to untreated cells=1;

FIG. 13 provides graphical results of studies conducted using the MNK inhibitor, cercosporamide (CSPM) to treat 3T3-L1 cells (cercosporamide (10 μM)). (A) Samples were analysed by RT-qPCR for the indicated mRNAs. Data are from three replicate experiments and are normalised to β2-microglobulin mRNA. (B) Samples were analysed for lipid accumulation;

FIG. 14 shows the results of: (A) 3T3-L1 cells treated with the MNK inhibitors, MNK-I1 and MNK-I2, at the indicated concentrations for 1 h and then samples analysed by immunoblot for phosphorylated and total elF4E. Similar data obtained in three replicate experiments. (B) Quantification of data for MNK-I1 and MNK-I2. (C) 3T3-L1 cells treated with MNK-I1 and MNK-I2 at the indicated concentrations for the indicated times and then samples were analysed by immunoblot for phosphorylated and total elF4E;

FIG. 15 shows graphical results for studies wherein: (A) 3T3-L1 cells were treated with a differentiation cocktail for the indicated times in the absence or presence of MNK-I1. Samples were analysed by RT-qPCR for the indicated mRNAs. Data are from three replicate experiments and are normalised to β2-microglobulin. Data for day 0 are set=1. (B) As (A) but cells were allowed to differentiate for nine days and then lysed and analysed for lipid (triglyceride) accumulation;

FIG. 16 provides the results of studies conducted on mouse embryonic fibroblasts (MEFs): MNK1-KO (A) or MNK2-KO (B). MEFs were treated with MNK-I1 or MNK12 at the indicated concentration for 1 h, and samples then analysed for P-eIF4E and total eIF4E. The graphs show combined data from three experiments. *, p<0.05 vs. untreated control; ****, p<0.0001, vs. untreated control. Pos, cells treated with 30 μM CGP57380; and

FIG. 17 shows graphical results for studies wherein: (A) 3T3-L1 cells were treated with a differentiation cocktail for the indicated times in the absence or presence of MNK-I2. Samples were analysed by RT-qPCR for the indicated mRNAs. Data are from three replicate experiments and are normalised to β2-microglobulin mRNA. Levels at day 0=1. (B) As (A) but cells were allowed to differentiate for nine days and then lysed and analysed for triglyceride.

### DETAILED DESCRIPTION

The present disclosure describes an investigation of the roles of MNK1 and MNK2 in mice consuming a normal chow diet (CD) or high fat diet (HFD) using mice in which MNK1 or MNK2 have been knocked out (MNK1-KO and MNK2-KO, respectively), and in a cellular model of adipocyte differentiation. It was found that inhibition of expression of MNK1 and/or MNK2, or reduction of the biological activity of MNK1 and/or MNK2 using an MNK inhibitor, can inhibit the effects of a high fat diet, including glucose intolerance, insulin resistance and lipogenesis.

The data provided herein demonstrates that MNK1 and MNK2 are expressed in adipose tissue, which is involved in the regulation of body metabolism by insulin. MNK2 mRNA is rapidly induced in a cell model of adipocyte differentiation, indicative of the involvement of this protein during adipogenesis. Notably, MNK2-KO mice were protected from HFD-induced fat gain observed in the WT/HFD mice. The size of the adipose cells of the MNK2-KO/HFD mice was observed to be no larger than their chow-fed counterparts. However, in comparison, the size of the adipose cells of the WT/HFD mice increased markedly compared to the WT mice on the chow diet (WT/CD). Both MNK1-KO/HFD and MNK2-KO/HFD mice were protected from indicators of insulin resistance, having reduced levels of circulating glucose and insulin, and a reduced HOMA-IR (an indicator of insulin resistance), better glucose tolerance, and reduced insulin resistance as indicated by a stronger response of the PKB signalling pathway (PKB phosphorylation) compared to the WT/HFD mice. Additionally, MNK2-KO/HFD mice showed decreased inflammation in adipose tissues. These results confirm the involvement of MNK1 and particularly MNK2 in mediating the adverse effects of a high fat diet.

In the presence of a small molecule inhibitor of both MNK1 and MNK2, CGP57380 (ie N3-(4-Fluorophenyl)-1H-pyrazolo-[3,4-d]pyrimidine-3,4-diamine; or 4-amino-3-(p-fluorophenylamino) pyrazolo[3,4-d]pyrimidine), the accumulation of lipid in an adipocyte cellular model was markedly inhibited. Further, CGP57380 inhibited expression of a number of genes required for adipocyte differentiation, indicating the important role of MNK1 and MNK2 in adipogenesis and lipid storage. These results demonstrate that reducing the biological function of MNK1 and/or MNK2 inhibits adipogenesis and lipid storage. Additionally, for cells differentiated in the presence of CGP57380, less glycerol was released in a lipolysis assay compared to cells differentiated in the absence of CGP57380, confirming decreased storage of triglyceride in the CGP57380-treated cells.

MNK1 and MNK2 are the only kinases known to phosphorylate eIF4E. MNK2-KO mice showed a substantial decrease in P-eIF4E compared to WT mice under both dietary conditions, whereas MNK1-KO mice showed no change in P-eIF4E on the chow diet, and only a slight reduction in P-eIF4E on the HFD, compared to WT animals. This indicates that MNK2 is the more active MNK isoform in adipose tissue.

Pre-diabetic subjects have impaired glucose metabolism. Two stages are recognised: (1) impaired fasting glucose (IFG) pre-diabetes, and (2) impaired glucose tolerance (IGT) pre-diabetes. Essentially, a standard oral glucose tolerance test (GTT) in human subjects will provide results that determine one of four diagnoses. The exact protocol for the GTT and the “cut off” glucose level used to diagnose glucose metabolism changes from country to country, but one commonly used example measures fasting blood glucose levels, and then a measured dose of glucose solution (usually containing 75 g glucose) is taken orally within a 5 minute time frame, and then blood glucose is measured again usually after two hours. Diagnosis is typically made as shown in Table 1. However, some small variation of the numbers shown in Table 1 are commonly cited. For the purposes of the present disclosure, the method may be used with pre-diabetic subjects having a fasting plasma glucose level from 5.5 mmol/l to 6.9 mmol/l (ie 100 mg/dl to 125 mg/dl).

TABLE 1
Typical blood glucose levels used to diagnose
pre-diabetes and diabetes

| | | Blood glucose level 2 hours |
|---|---|---|
| Diagnosis | Fasting blood glucose level | after GTT test |
| Normal glucose levels | <6.1 mmol/L | <7.8 mmol |
| Impaired Fasting Glucose pre- | ≥6.1 mmol/L < 7.0 mmol/L | <7.8 mmol |
| diabetes | | |
| Impaired Glucose Tolerance | ≥6.1 mmol/L < 7.0 mmol/L | ≥7.8 mmol < 11.1 mmol/L |
| pre-diabetes | | |
| Type 2 diabetes | ≥7.0 mmol/L | ≥11.1 mmol/L |

Many pre-diabetic subjects remain pre-diabetic for between, for example, 3 and 15 years without any significant change in symptoms; whereas for others, the symptoms worsen more quickly and they progress through to type 2 diabetes. Importantly, while the diagnosis of pre-diabetes is considered a high risk factor for eventually developing type 2 diabetes, the two stages of pre-diabetes are distinct from each other and from type 2 diabetes, as not all subjects diagnosed with impaired fasting glucose pre-diabetes will progress to impaired glucose tolerance pre-diabetes or type 2 diabetes. Likewise, not all subjects with impaired glucose tolerance pre-diabetes will progress to type 2 diabetes.

The method of the present disclosure may prevent and/or delay progression from pre-diabetes to type 2 diabetes.

Thus, in a first aspect, the present disclosure provides a method of treating a subject with impaired fasting glucose pre-diabetes to prevent and/or delay progression of the pre-diabetes to type 2 diabetes, said subject characterised by having a fasting plasma glucose level from 5.5 mmol/l to 6.9 mmol/l, wherein said method comprises administering to the subject a therapeutically effective amount of at least one mitogen-activated protein kinase-interacting kinase (MNK) inhibitor, wherein said MNK inhibitor reduces the biological activity of MNK2 and, optionally, MNK1.

The method of the present disclosure may also prevent progression of pre-diabetes at the impaired fasting glucose (IFG) stage to the impaired glucose tolerance (IGT) stage. In addition, the method may also avoid or reduce symptoms and/or effects associated with pre-diabetes such as obesity, increase in body weight, increase in adipose tissue weight, increase in adipose tissue inflammation, increase in circulating glucose and/or resistance to glucose, increase in circulating insulin and/or resistance to insulin.

As mentioned above, pre-diabetic subjects are frequently over-weight, and the condition is generally associated with an unhealthy diet, such as a high fat diet. The term “high fat diet” is used herein to refer to diets that typically have a higher percentage of calories (obtained from the fat in the diet) compared to a normal diet. The fat in the diet may include all types of dietary fat, whether animal or vegetable and whether monounsaturated, polyunsaturated, saturated, etc. Thus, the high fat diet has a higher caloric content than a normal diet. In an embodiment, a pre-diabetic subject treated in accordance with the disclosed method has a high fat diet comprising a diet having equal to or greater than 30% of total energy from fat. In another embodiment, a pre-diabetic subject treated in accordance with the disclosed method has a high fat diet comprising a diet having equal to or greater than 35%, 40%, 45%, 50%, 55%, 60%, or 65% of total energy from fat. Additionally or alternatively, a pre-diabetic subject treated in accordance with the disclosed method has a high fat diet characterised by a higher total energy value than a normal diet. For example, recommended energy intake for a normal diet for middle-aged adults is shown in Table 2.

TABLE 2
Recommended energy intake in a normal diet in adults

| | | | | | |
|---|---|---|---|---|---|
| Height | Age | Weight | Activity | kJ/day | Calories/day |

Females

| | | | | | |
|---|---|---|---|---|---|
| 1.6 m | 31-50 | 60 kg | Sedentary* | 7600 | 1800 |
| | | | Moderate** | 9800 | 2350 |

Males

| | | | | | |
|---|---|---|---|---|---|
| 1.9 m | 31-50 | 70 kg | Sedentary* | 9900 | 2400 |
| | | | Moderate** | 12700 | 3000 |

*Sedentary - seated work with little or no strenuous leisure activity.
**Moderate - standing or walking work, or sedentary work with regular exercise of at least 30 minutes.

Those skilled in the art will appreciate that recommended energy intake values indicate the energy intake levels of normal diets. However, these values will vary according to age, gender, height, and activity levels. Accordingly, a high fat diet may have approximately the same energy intake as a normal diet, or otherwise have a higher total energy intake than a normal diet, for example, 10-30%, 20-40%, 30-50% or 30-60% higher total energy intake compared to a normal diet. Accordingly, a pre-diabetic subject treated in accordance with the disclosed method may have a high fat diet with an energy intake value that is at least 5%, 10%, 15%, 20%, 25%, 30%, 35%, 40%, 45%, 50%, 55%, 60%, 65%, 70%, 75%, 80%, 90%, 100%, 125%, 150% or 200% higher than the energy intake value of a normal diet.

The HFD-fed male mouse is a widely-used and accepted model for studying the effects of a high fat diet (Panchal et al., 2011). These animals show many of the same responses, in terms of increased fat tissue, adipose tissue inflammation and defects in insulin responses (eg impaired glucose tolerance), to those associated with obesity-associated metabolic syndrome in humans (Hariri and Thibault, 2010). As an example, a high fat diet for mice may comprise 45% kcal fat, 20% kcal protein, 35% kcal carbohydrate compared to a standard chow diet comprising 7% kcal fat, 18% kcal protein, 75% kcal carbohydrate. However, those skilled in the art will appreciate that a high fat diet for mice may range from 32% to 60% of energy from fat.

As disclosed herein, both MNK1-KO/HFD and MNK2-KO/HFD mice had higher glucose tolerance compared to WT/HFD animals. In this test, animals were fasted overnight before receiving an intraperitoneal injection of 2 g/kg glucose, and glucose was measured in the blood before and at several time points up to two hours following glucose administration. Both MNK1-KO/HFD and MNK2-KO/HFD mice had lower glucose levels compared to WT/HFD animals at every time point measured. This is particularly relevant as it demonstrates that on the same high fat diet, having a reduced MNK1 and/or MNK2 biological activity increases glucose tolerance, and accordingly, indicates that reducing MNK bioactivity would assist in the treatment or prevention of pre-diabetes. Accordingly, in an embodiment, the condition to be treated or prevented by the MNK inhibitor is pre-diabetes.

The human MNKs comprise a group of four proteins derived from two genes (Gene symbols: MKNK1 and MKNK2) by alternative splicing. MNK1a/1b differ at their C-termini, as do MNK2a/2b. In each case, the a-form possesses a longer C-terminal region than the b-form, which lacks the MAP kinase-binding region. The N-termini of all forms contain a polybasic region which binds importin a and the translation factor scaffold protein eukaryotic initiation factor 4G (eIF4G). The catalytic domains of MNK1a/b and MNK2a/b share three unusual features: two short inserts and a DFD feature where other kinases have DFG. MNK isoforms differ markedly in their activity and regulation, and in subcellular localisation. The best characterised MNK substrate is eIF4E. The cellular role of eIF4E phosphorylation remains unclear; it may promote export of certain mRNAs from the nucleus. Other MNK substrates bind to AU-rich elements that modulate the stability/translation of specific mRNAs. MNKs may also control the production of inflammatory mediators and signalling from tyrosine kinase receptors, as well as cell proliferation or survival.

The sequences for the human MNKs can be found as follows: Human MNK1 mRNA and amino acid sequence: Genbank Accession No. AB000409.1; Human MNK1 amino acid sequence variant: Genbank Accession No. NM-003684.2; Human MNK2a mRNA sequence: Genbank Accession No. AF237775; Human MNK2b mRNA and amino acid sequence: Genbank Accession No. AF237776, and variant of the nucleic acid sequence labeled NM-17572.2. The term “GenBank Accession number” relates to National Center for Biotechnology Information (NCBI) GenBank database entries (Benson et al, 2000). The information disclosed at each of the above GenBank Accession numbers is hereby incorporated by reference.

The term “MNK inhibitor” as used herein is intended to refer to any compound that reduces the biological activity of MNK1 (including reducing the biological activity of one or both of splice variants MNK1 a and MNK1b) and/or MNK2 (including reducing the biological activity of one or both of splice variants MNK2a and MNK2b), including by reducing the amount of MNK1 and/or MNK2 expressed. For example, the MNK inhibitor may be a small organic molecule (also referred to as a “small molecule” herein), a peptide antagonist, an inhibitory antibody or fragment thereof, an interfering nucleotide molecule, an aptamer or other MNK inhibitor that inhibits the expression or biological activity as would be understood by those skilled in the art. The biological activity of MNK1 and MNK2 can be measured in a variety of ways, for example, by measuring phosphorylation of its substrate eIF4E to P-eIF4E using any suitable method known to those skilled in the art, for example, by performing an immunoblot or ELISA utilising a specific anti-P-eIF4E antibody, for example, the anti-phospho eIF4E (Ser209) antibody distributed by Merck Millipore, Catalogue No. 07-823 (Merck Millipore, Billerica, Mass., United States of America). Accordingly, a relative decrease in the amount of P-eIF4E in a sample containing the MNK inhibitor as compared to a corresponding sample in the absence of the MNK inhibitor demonstrates that the biological activity of the MNK has decreased. Those skilled in the art will be aware of how to conduct suitable controls when measuring P-eIF4E levels. The MNK1 and/or MNK2 bioactivity can otherwise be measured using a cellular assay that can detect levels of MNK1 and/or MNK2 bioactivity. Antibodies that specifically detect phosphorylated MNK could also be used, for example, anti-P-MNK1 (Phospho-Mnk1 (Thr197/202) Antibody #2111; Cell Signaling Technology Inc, Danvers, Mass., United States of America). Additionally, those skilled in the art will appreciate that the MNK inhibitors should be safe for use for its intended purpose.

In some embodiments, the MNK inhibitor may preferably show selectivity and/or increased inhibitory potency to MNK2. A simple test for assessing the selectivity of an MNK inhibitor can be conducted by treating suitable cells from animals in which either MNK1 or MNK2 has been knocked out (eg mouse embryonic fibroblasts (MEFs) from MNK1-KO and MNK2-KO mice) with the MNK inhibitor. Since, in MNK1-KO MEFs, eIF4E phosphorylation depends on MNK2, and vice versa, monitoring the effect of different concentrations of an MNK inhibitor of interest on P-eIF4E levels in MNK-KO MEFs reports on the ability of that compound to inhibit MNK2 (MNK1-KO MEFs) or MNK1 (MNK2-KO MEFs). Thus, by comparing the data for the two kinds of cells, the degree of selectivity that the tested MNK inhibitor shows towards MNK1 or MNK2 can be determined.

The MNK inhibitor may be selected from the group consisting of a small organic molecule, a peptide inhibitor, an inhibitory antibody or fragment thereof, an inhibitory nucleotide molecule in including an interfering RNA molecule, and an aptamer. MNK inhibitors may include those already known in the art. In an embodiment, combinations of MNK inhibitors may be used.

In an embodiment, the MNK inhibitor is a small organic molecule. A number of examples of suitable small molecule inhibitors of MNK1 and/or MNK2 are described below.

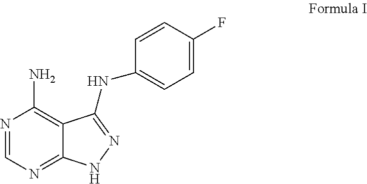 [SMILES: Nc1ncnc2[nH]nc(Nc3ccc(F)cc3)c12]

Formula I shows CGP57380 (N3-(4-Fluorophenyl)-1H-pyrazolo-[3,4-d]pyrimidine-3,4-diamine; or 4-amino-3-(p-fluorophenylamino) pyrazolo[3,4-d]pyrimidine; WO 03/037362; Chrestensen et al., 2007; Buxade et al., 2005; Worch et al., 2004, Rowlett et al., 2008), which is commercially available. It is an inhibitor of both MNK1 and MNK2 (Hou et al., 2012).

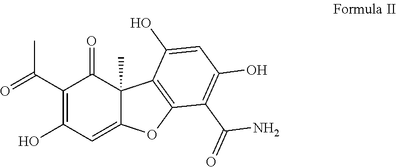 [SMILES: CC(=O)C1=C(O)C=C2Oc3c(C(N)=O)c(O)cc(O)c3[C@]2(C)C1=O]

Formula II shows cercosporamide ((9aS)-8-Acetyl-9,9a-dihydro-1,3,7-trihydroxy-9a-methyl-9-oxo-4-dibenzofurancaboxamide; Sussman et al., 2004), which is commercially available.

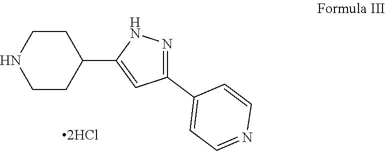 [SMILES: c1cc(-c2cc(C3CCNCC3)[nH]n2)ccn1]

Formula III shows ETP 45835 dihydrochloride (4-[5-(4-Piperidinyl)-1H-pyrazol-3-yl]pyridine dihydrochloride; Oyarzabal et al., 2010), which is commercially available.

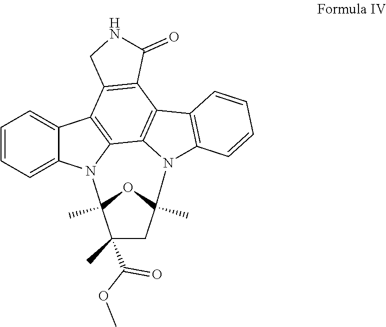

Formula IV shows CGP052088 (Tschopp et al., 2000), a derivative of staurosporine.

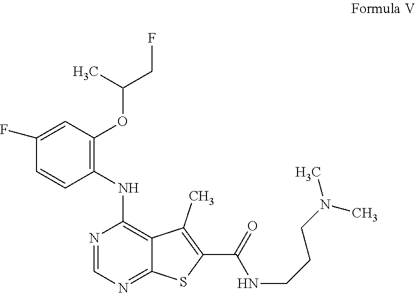 [SMILES: Cc1c(C(=O)NCCCN(C)C)sc2ncnc(Nc3ccc(F)cc3OC(C)CF)c12]

Formula V shows MNK-I1 (4-(2-(2-fluoropropoxy)-4-fluorophenylamino)-N-(3-(dimethylamino)propyl)-5-methylthieno[2,3-d]pyrimidine-6-carboxamide; Beggs et al. 2015).

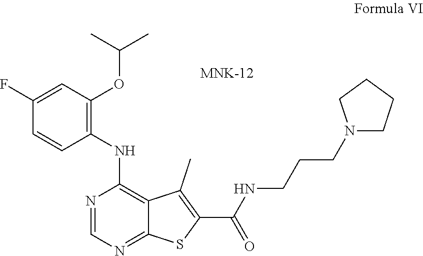

Formula VI shows MNK-12 (4-(2-isopropoxy)-4-fluorophenylamino)-N-(3-(pyrrolidin-1-yl)propyl)-5-methylthieno[2,3-d]pyrimidine-6-carboxamide).

The IC₅₀ details regarding some of these compounds are shown in Table 3.

TABLE 3
Half maximal inhibitory concentration (IC₅₀) values
of small molecule MNK inhibitors

| Compound | MNK1 IC₅₀ | MNK2 IC₅₀ | Reference |
|---|---|---|---|
| CGP57380 | 0.7 μM | 0.8 μM | Buxade et al., 2008 |
| | | | Knauf et al., 2001 |
| cercosporamide | 0.116 μM | 0.11 μM | Konicek et al., 2011 |
| ETP 45835 | 0.575 μM | 0.646 μM | Oyarzabal et al., 2010 |
| dihydrochloride | | | |
| CGP052088 | 70 nM | | Tschopp et al., 2000 |
| Mnk-I1 | 0.023 μM | 0.016 μM | Beggs et al., 2015 |

In some embodiments, a small organic molecule-type MNK inhibitor for use in the method of the present disclosure will be selected from the group of compounds known as “ATP competitors” or “type I kinase inhibitors” (Hou et al., 2012; Liu and Gray, 2006) which interact with the ATP binding domain of MNKs (eg CGp57380 and cercosporamide; Hou et al., 2012).

In some embodiments, a small organic molecule-type MNK inhibitor for use in the method of the present disclosure will be selected from the group of thienopyrimidine compounds. Examples of such compounds include those described in WO 06/136402 and WO 2007/115822; thienopyrimidinyl derivatives described in Teo et al., 2015 (including a MNK2 selective inhibitor), and thienopyrimidinyl derivatives containing a substituted alkyl group such as those described in WO 2011/104340 and U.S. Pat. No. 8,633,201; the content of all of specifications referred to in this paragraph are hereby incorporated by reference in their entirety. Preferred examples of suitable thienopyrimidine compounds may include those described in WO 2011/104340 (the content of which is hereby incorporated by reference in its entirety) and the specific compounds mentioned above, MNK-I1 and MNK-I2. Particularly preferred examples of suitable thienopyrimidine compounds may be of the general formula shown below:

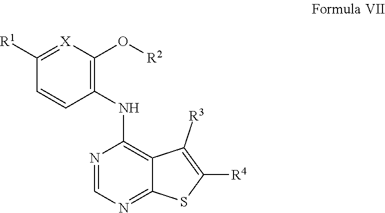

wherein: X is selected from CH and N; R¹ is H, a halogen atom (such as F), CN, a C₁₋₆ alkyl group (preferably a C₁₋₃ alkyl group) or a CONH₂ group; R² is a straight-chained or branched C₁₋₆ alkyl group (preferably a C₁₋₃ alkyl group) which may be independently substituted with a halogen atom(s) (such as F) or one or two trihalogen-methyl (eg a trifluoromethyl), tetrahydropyranyl, cyclopropyl, H₂N—CO—, R⁵NHCO— or (R⁵)₂N—CO— groups, wherein the cyclopropyl group may be substituted with one or two halogen atom(s) (such as F) or —CH₂—CN, and wherein, in the case that the compound comprises a (R⁵)₂N—CO— group, the two R⁵ groups may form together with the N atom to which they are attached, a 4- to 8-membered ring, in which a carbon atom may be replaced by an O, S, SO or SO₂ and/or may be substituted with OH, NH₂, N(C₁₋₃-alkyl)₂, NH(C₁₋₃ alkyl), CF₃, C₁₋₃-alkyl group, or R² is a straight-chained or branched C₂₋₆ alkyl group which is independently substituted in position 2 to 6 with one or two OH, C₁₋₃ alkoxy, amino, CN, R⁶NH—, (R⁶)₂N—, R⁶OCONH—, R⁶CONH—, R⁶SO₂NH— or R⁶NHCONH— groups, wherein R⁶ is a C₁₋₅ alkyl group (preferably a C₁₋₄ alkyl group such as CH₃, i-Pr and t-Bu), each optionally substituted with one CF₃, NH₂, NH(C₁₋₃ alkyl), N(C₁₋₃ alkyl)₂ or CH₃O-group, and wherein the hydrogen atoms of any of the above-mentioned NH moieties may be replaced by CH₃; R³ is H or a C₁₋₃ alkyl group (preferably CH₃); and R⁴ is selected from a carboxy group, C₁₋₃ alkoxy-carbonyl, —CONH₂, —CONHR⁷, —CONH—OR⁷, —CONH—SO₂R⁷ and a —CO—NH-L-R⁷ group, wherein L is a —(CH₂)ₙ— (where n is 2, 3 or 4) or C₃-6 branched chain alkyl residue (eg —CH₂—C(CH₃)₂—CH₂—), —CH₂—C≡C—CH₂—, or

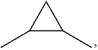

and R⁷ is selected from OH, —NH₂, —NHR⁸, —N(R⁸)₂, —NH—CO₂R⁸ or a 3- to 6-membered cyclic ring (eg phenyl or morpholine group or a cyclic amine (eg pyrrolidine or piperidine), and R⁸ is C₁₋₃ alkyl (preferably CH₃), or a tautomer, enantiomer or salt thereof. More particularly, preferred compounds of formula (VII) are those wherein X is as defined above (but preferably CH₂), R¹ is H or, more preferably, F, R² is a straight-chained or branched C₁₋₃ alkyl group which may be independently substituted with a halogen atom(s) (such as F) or one or two trihalogen-methyl (eg a trifluoromethyl), tetrahydropyranyl, cyclopropyl, H₂N—CO—, R⁵NHCO— or (R⁵)₂N—CO— groups, R³ is CH₃, and R⁴ is selected from a carboxy group, C₁₋₃ alkoxy-carbonyl, —CONH₂, —CONHR⁷, —CONH—OR⁷, —CONH—SO₂R⁷ and a —CO—NH-L-R⁸ group wherein L is a —(CH₂)ₙ— (where n is 2 or 3) or C₃₋₆ branched chain alkyl residue (eg —CH₂— C(CH₃)₂—CH₂—), wherein R⁷ is selected from OH, —NH₂, —NHR⁸ and —N(R⁸)₂, —NH—CO₂R⁸ (where R⁸ is C₁₋₃ alkyl and preferably CH₃) and a 3- to 6-membered cyclic ring such a cyclic amine (preferably pyrrolidine), or a tautomer or salt thereof. Even more preferred compounds of formula (VII) may be those wherein X is as defined above (but preferably CH₂), R¹ is F, R² is a straight-chained or branched C₁₋₃ alkyl group which may be independently substituted with a halogen atom(s) (such as F), R³ is CH₃, and R⁴ may be a —CONHR⁷ or a —CONH—OR⁷ but is more preferably a —CO—NH-L-R⁸ group wherein L is —(CH₂)ₙ— (where n is 2 or 3) or C₃₋₆ branched chain alkyl residue (eg —CH₂— C(CH₃)₂—CH₂—), wherein R⁷ is selected from OH, —NH₂, —NHR⁸ and —N(R⁸)₂, —NH—CO₂R⁸ (where R⁸ is C₁₋₃ alkyl and preferably CH₃) and a 3- to 6-membered cyclic ring such a cyclic amine (preferably pyrrolidine), or a tautomer or salt thereof.

Other small molecules that reduce the biological activity of MNK1 and/or MKN2 may also be suitable for use as described herein. Examples of such compounds include those described in EP 0819129, pyridine and pyrazine derivatives such as those described in WO 2007/147874, and 8-heteroarylpurine compounds such as those described in U.S. Pat. No. 7,951,803; all of which are hereby incorporated by reference. However, those skilled in the art will appreciate that other small molecules are suitable for use in the method of the present disclosure, providing they inhibit MNK biological activity and/or expression as described herein, and are considered safe for their purpose. Methods for screening small compounds for MNK inhibitory function are described in U.S. Pat. No. 8,633,201. Further, considerations for designing additional small organic molecule MNK inhibitors are described in Hou et al. (2012), the content of which is hereby incorporated by reference in its entirety.

In an embodiment, the MNK inhibitor is a peptide inhibitor.

Peptide inhibitors are peptides or proteins or fragments thereof that are capable of binding with a target, such as MNK 1 and/or MNK2, in such a manner that the biological activity of MNK1 and/or 2 is reduced. Peptide inhibitors may be naturally occurring MNK binding partners or be other peptides, whether derived from a natural source or artificially synthesised. A large number of peptide libraries are known, and such libraries can be screened by a number of techniques known to those skilled in the art. Novel peptide inhibitors of MNK1 and/or MKN2 can be identified using such methods, and produced using techniques well known to those skilled in the art.

In an embodiment, the MNK inhibitor may be an inhibitory antibody or fragment thereof, for example, an antibody that is specific for MNK that may be used directly to inhibit or antagonise the biological activity of MNK1 and/or MNK2. Inhibitory antibodies may alternatively be known as neutralising antibodies.

Suitable antibodies may be generated using methods that are well known to those skilled in the art, and then screened to identify antibodies that have MNK inhibitory properties, that is, that reduced the biological activity of MNK1 and/or MNK2. Such antibodies may include, but are not limited to, polyclonal, monoclonal, chimerical, single chain, Fab fragments, and fragments produced by a Fab expression library. Methods of making antibodies that have MNK inhibitory properties are well known to those skilled in the art.

In an embodiment, the MNK inhibitor may be an inhibitory aptamer, for example, an aptamer that is specific for MNK that may be used directly to inhibit or antagonise the biological activity of MNK1 and/or MNK2. An aptamer is an oligonucleotide or peptide molecule that binds to a specific target molecule, with oligonucleotide molecules consisting of (usually) short strands of oligonucleotides, and peptide aptamers typically consisting of a short variable peptide domain (or loop), attached at both ends to a protein scaffold. Aptamers are capable of inhibiting proteins, such as MNK1 and MNK2, with high affinity and specificity. Any suitable method of producing aptamers and screening them for the desired inhibitory action may be used to produce a MNK inhibitor. Examples of such methods for nucleotide aptamers are described in U.S. Pat. No. 7,960,102 and WO 91/19813; however, other methods may also be suitable. Peptide aptamers can also be selected from combinatorial peptide libraries constructed by phage display and other surface display technologies such as mRNA display, ribosome display, bacterial display and yeast display. Further examples of methods for producing inhibitory peptide aptamers are described in WO 2012/096978.

In an embodiment, the MNK inhibitor may be an inhibitory nucleotide molecule. Double-stranded or single-stranded interfering RNA molecules can induce sequence-specific degradation of the mRNA transcripts of a given gene, thereby inhibiting translation of the mRNA into protein as is well known to those skilled in the art. Antisense molecules to the polynucleotide encoding MNK may alternatively be used in situations in which it would be desirable to block the transcription of the mRNA. Accordingly, inhibitory nucleotide molecules may be used to inhibit MNK biological activity, by reducing the amount of MNK protein produced. Such technology is now well known to those skilled in the art, and sense or antisense oligomers or larger fragments, can be designed from various locations along the coding or control regions of sequences encoding MNK. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding MNK. Genes encoding MNK can be turned off by transforming a cell or tissue with expression vectors which express high levels of polynucleotide or fragment thereof which encode MNK. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system. Inhibitory nucleotide molecules may be prepared by any method known to those skilled in the art for the synthesis of nucleic acid molecules.

The MNK inhibitor may be administered to the subject by any of the accepted modes for enteral administration such as oral or rectal, or by parenteral administration such as subcutaneous, intramuscular, intravenous and intradermal routes, or by any other suitable route such as the intranasal route. Injection can be bolus or via constant or intermittent infusion. In an embodiment, the MNK inhibitor is administered orally. Accordingly, the MNK inhibitor may be formulated in an oral dosage form such as, for example, a capsule, tablet, caplet, granules or powders (which may be suspended or dissolved in water to provide a beverage), or as a fortified food.

The MNK inhibitor can be administered in any form or mode which makes it bioavailable. Those skilled in the art of preparing formulations can readily select the proper form and mode of administration depending upon the particular characteristics of the MNK inhibitor selected, the condition to be treated, the stage of the condition to be treated and other relevant circumstances (see Remingtons Pharmaceutical Sciences, 19th edition, Mack Publishing Co. (1995) for further information). In an embodiment, the formulation may optionally be combined with a pharmaceutically or veterinary-acceptable filler, carrier, diluent and/or excipient. The filler, carrier, diluent or excipient may be any suitable substance known to those skilled in the art, for example, dicalcium phosphate dibasic (DCPD), dibasic calcium phosphate, magnesium stearate, starches, sugars, lactose, sucrose, glucose, mannitol, sorbitol, calcium carbonate, cellulose, cellulose derivatives or modified cellulose such as microcrystalline cellulose, hydroxypropyl cellulose, methyl cellulose, alcohols like xylitol, sorbitol or maltitol, water, alcohol, gelatin, polyvinylpyrrolidone, sodium starch glycolate, and/or fumed silica absorbent. Preferably, the filler, carrier, diluent or excipient may be magnesium stearate, DCPD, sodium starch glycolate, fumed silica absorbent, or a combination thereof.

The MNK inhibitor may be used or administered in combination with one or more additional drug(s) for the treatment of the conditions described herein. The components can be administered in the same formulation or in separate formulations. If administered in separate formulations, the MNK inhibitors may be administered sequentially (ie sequentially in any order within, for example, seconds or minutes or even hours (eg 2 to 48 hours)) or simultaneously with the other drug(s).

In addition to being able to be administered in combination with one or more additional drugs, the MNK inhibitor may be used in a combination therapy. When this is done, the MNK inhibitors are typically administered in combination with each other. Thus, one or more of the MNK inhibitors may be administered either simultaneously (as a combined preparation) or sequentially (ie sequentially in any order within, for example, seconds or minutes or even hours (eg 2 to 48 hours)) in order to achieve a desired effect. This is especially desirable where the therapeutic profile of each MNK inhibitor is different such that the combined effect of the two drugs provides an improved therapeutic result.

If desired, and for more effective distribution, the MNK inhibitor can be incorporated into slow release or targeted delivery systems such as polymer matrices, liposomes and microspheres.

A “therapeutically effective amount” of the MNK inhibitor may vary depending upon, for example, the particular selected MNK inhibitor or combination of MNK inhibitors employed, the mode of administration, the particular condition being treated, and the desired outcome. It will also depend upon the stage and severity of the condition, the subject to be treated including the age and physical condition of the subject, the nature of concurrent therapy, if any, and like factors well-known to the medical practitioner. For prophylactic (preventative) applications, it is generally that amount sufficient to delay the onset of, inhibit the progression of, or halt altogether the particular condition sought to be prevented. For therapeutic applications, it is generally that amount sufficient to achieve a medically desirable result.

The determination of a therapeutically effective amount is well within the capability of those skilled in the art. For any compounds, the therapeutically effective amount can be estimated initially either in cell culture assays (eg of pre-adipocyte cell lines), or in animal models, usually mice, rabbits, dogs or pigs. The animal model may also be used to determine the appropriate concentration range and route of administration. Such information can then be used to determine useful dose amounts and routes for administration in humans. Therapeutic efficacy and any toxicity may be determined by standard pharmaceutical procedures in cell cultures or experimental animals, for example through determination of the ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage amounts for human use. The dosage amount contained in such compositions is preferably within a range of circulating concentrations that include the ED50 with little or no toxicity. The dosage amount varies within this range depending upon the dosage form employed, sensitivity of the patient, and the route of administration. The exact dosage amount and therapeutically effective amount to be used will be determined by the practitioner, in light of factors related to the subject that requires treatment. The dosage amount and route/frequency of administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Pharmaceutical compositions may be administered several times a day, once a day, every 3 to 4 days, every week, or once every two weeks depending on half-life and clearance rate of the particular formulation and/or active moiety. The therapeutically effective amount may vary from 0.1 to 100,000 mg, up to a total dose of about 1 g, depending upon the route of administration. In one example, a therapeutically effective amount may generally be from about 1 to 2000 mg/day, preferably from about 10 to about 1000 mg/day, and most preferably from about 10 to about 500 mg/day, which may be administered in one or multiple doses.

It is intended that any or all of the above described embodiments of the method of the disclosure could readily be used in other aspects of the disclosure including the following:

Another aspect of the disclosure provides the use of a therapeutically effective amount of an at least one MNK inhibitor for treating a subject with impaired fasting glucose pre-diabetes to prevent and/or delay progression of the pre-diabetes to type 2 diabetes, said subject characterised by having a fasting plasma glucose level from 5.5 mmol/l to 6.9 mmol/l, wherein said MNK inhibitor reduces the biological activity of MNK2 and, optionally, MNK1.

A further aspect of the disclosure provides the use of an at least one MNK inhibitor that reduces the biological activity of MNK2 and, optionally, MNK1 in the manufacture of a medicament for treating a subject with impaired fasting glucose pre-diabetes to prevent and/or delay progression of the pre-diabetes to type 2 diabetes, said subject characterised by having a fasting plasma glucose level from 5.5 mmol/1 to 6.9 mmol/l.

### EXAMPLES

### Example 1 MNK1 and MNK2 Mediate the Adverse Effect of a High Fat Diet, and Inhibiting MNK1 and MNK2 Biological Function Reduces these Effects

### Materials and Methods

Materials and Chemicals

All cell culture solutions and supplements were purchased from Life Technologies (Carlsbad, Calif., United States of America). Reagents for SDS-PAGE were purchased from Bio-Rad Laboratories, Inc. (Hercules, Calif., United States of America). For the adipogenesis experiment, insulin, dexamethasone, IBMX and rosiglitazone were obtained from Sigma-Aldrich Corporation (St Louis, Mo., United States of America). For macrophage polarisation, lipopolysaccharide (LPS) was purchased from Sigma-Aldrich, interleukin (IL)-4 and interferon (IFN) γ were purchased from Peprotech (Rocky Hill, N.J., United States of America). The MNK inhibitor CGP57380 was obtained from Abcam PLC (Cambridge, United Kingdom).

Animal Use and Diet

MNK1-KO and MNK2-KO mice were created on a C57BL/6J background and kindly provided by Dr Rikiro Fukunaga (Osaka University, Japan; Ueda et al., 2004). Four week old male wild-type (WT), MNK1-KO or MNK2-KO mice were kept under a 12-h light/dark cycle (lights on at 07:00 h) and at a constant temperature of 22±2° C. with food and water available ad libitum. They were allocated to either a high fat diet (HFD; 45% kcal fat, 20% kcal protein, 35% kcal carbohydrate; Special Dietary Services, Essex, United Kingdom) or a standard chow diet (C; 7% kcal fat, 18% kcal protein, 75% kcal carbohydrate; Special Dietary Services) for 20 weeks. The fat in the diets was provided by lard (17.89% w/w) and soya oil (4.32% w/w). Nutritional values for the diets are shown in Table 4.

TABLE 4
Nutritional value of High Fat Diet and Standard Chow Diet

| Energy per kg of feed | High fat diet | Standard chow diet |
|---|---|---|
| Gross energy (MJ/kg) | 20.30 | 14.74 |
| Digestible energy (MJ/kg) | 18.61 | 11.90 |
| Metabolisable energy (MJ/kg) | 17.19 | 10.74 |
| Atwater Fuel Energy (Kcal/kg) | 4568.36 | 3289.41 |

Metabolic Studies

After 15 weeks on either the chow or HFD, a glucose tolerance test (GTT) was performed after an overnight fast. Fasting glucose concentration was measured from whole blood obtained from the tail vein before the mice were intraperitoneally (ip) injected with D-glucose (2 g/kg mouse body weight; Baxter Healthcare Ltd, Adelaide, Australia)), and blood glucose concentrations were measured from the tail vein at 15, 30, 60 and 120 min post ip injection using an Aviva Accu-Chek glucometer (Roche Diagnostics, Risch-Rotkreuz, Switzerland).

To test for insulin resistance, a separate cohort of mice was fasted overnight and fasting glucose concentration in whole blood obtained from the tail was measured before ip insulin injections (0.75 U/kg mouse body weight; Actrapid, Novo Nordisk, Bagsvaerd, Denmark). Blood glucose concentrations were then measured from the tail vein at 15 and 30 min post ip injection. Mice were immediately sacrificed after taking the glucose reading 30 min post ip injection and tissues were frozen immediately for analysis by Western blotting to measure downstream insulin signalling.

Measurements of plasma insulin, IL-5 and IL-10 were performed by enzyme-linked immunosorbent assay (ELISA) carried out by Core Biochemical Assay Laboratory (CBAL) in Cambridge, United Kingdom.

Western Blotting

Tissues were harvested in RIPA lysis buffer containing 50 mM TrisHCl, pH 7.4, 150 mM NaCl, 1% Triton X-100, 0.1% sodium deoxycholate, 0.1% sodium dodecyl sulfate, 1 mM ethylenediaminetetraaacteic acid (EDTA), 50 mM β-glycerolphosphate, 0.5 mM NaVO3, 0.1% 2-mercaptoethanol and protease inhibitors (Roche). After lysis, insoluble material was removed by centrifugation at 12,000 g for 10 min at 4° C. Protein content was determined by the Bradford protein assay (Bio-Rad). Immunoblotting was performed as described in Liu et al. (2014). Blots were visualised using a LI-COR Odyssey® Quantitative Imaging System. Primary antibodies as indicated in Table 5 were from Cell Signaling Technologies, except for anti-P-eIF4E (Merck Millipore). Secondary antibodies were obtained from Thermo Fisher Scientific Inc. (Waltham, Mass., United States of America) and used at 1:20,000 dilution.

TABLE 5
Antibodies used in experiments

| | | Antibody | |
|---|---|---|---|
| Antibodies | Species Reactivity | type | Secondary |
| P-eIF4E S209 | Mouse, Rat, Human | Monoclonal | Rabbit |
| eIF4E | Human, Mouse, Rat, Monkey, Mink, Zebrafish | Polyclonal | Rabbit |
| P-PKB S473 | Human, Mouse, Rat, Hamster, | Polyclonal | Rabbit |
| | D. melanogaster, Bovine, Dog, Pig | | |
| P-PKB T308 | Human, Mouse, Rat, Hamster, | Polyclonal | Rabbit |
| | D. melanogaster, Bovine, Dog, Pig | | |
| MNK1 | Human, Mouse | Monoclonal | Rabbit |
| Actin | Mouse, Rat, Human | Monoclonal | Mouse |
| α-Tubulin | Human, Mouse, Rat, Chicken, Bovine, yeast | Monoclonal | Mouse |
| PPARγ | Mouse, Human | Monoclonal | Rabbit |
| P-ERK | Human, Mouse, Rat, Hamster, Monkey, Mink, | Monoclonal | Rabbit |
| T202/Y204 | Drosophila, Zebrafish, Bovine, Dog, Pig, | | |
| | Saccharomyces cerevisiae | | |
| P-rpS6 S240/44 | Mouse, Rat, Human, Monkey | Monoclonal | Rabbit |
| P-4E-BP1 | Human, Mouse, Rat, Monkey, D. melanogaster | Monoclonal | Rabbit |
| T37/46 | | | |
| Glut 4 | Mouse, Rat, Human | Monoclonal | Mouse |

Gene Expression Analysis

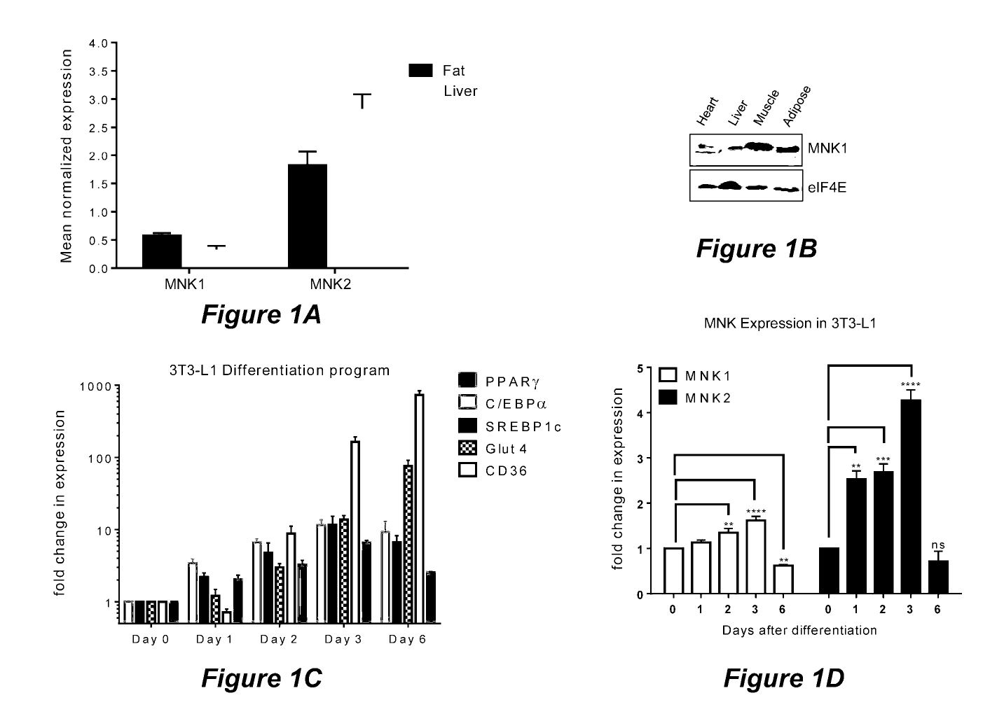

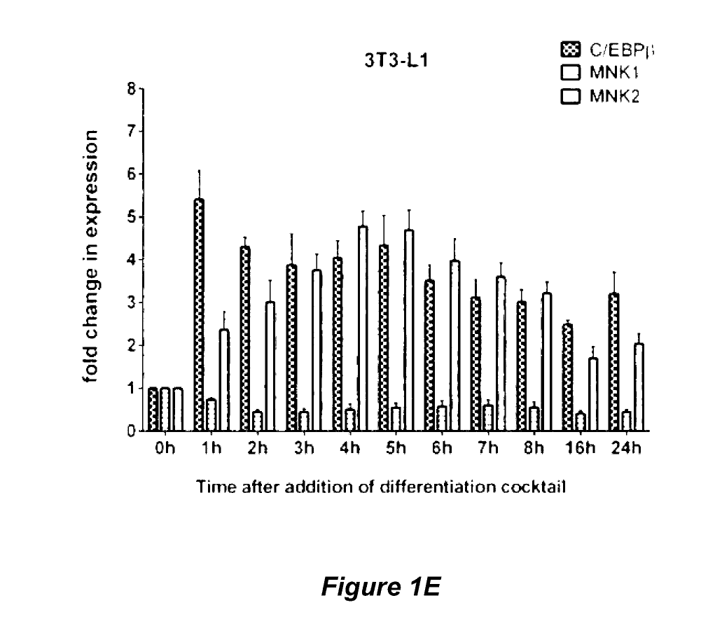

Total RNA was isolated from tissues (frozen at the time of sampling) using Trizol reagent (Sigma-Aldrich) according to the manufacturer's instructions. RNA concentrations were determined by absorbance at 260 nm, while quality and integrity were evaluated via 260/230 and 260/280 ratios using a NanoDrop spectrophotometer (Thermo Fisher Scientific). RT-real time PCR amplification was carried out using the ImProm-II Reverse Transcription System (A3800; Promega, Madison, Wis., United States of America) with random primers following the manufacturer's protocol. Subsequently, real-time quantitative (q) PCR was performed using primer sequences described in Supplemental Table S1 of Moore CEJ et al., 2016, the content of which is hereby incorporated by reference in its entirety). The comparative C_{T} method was used to measure the amplification of target mRNA levels compared with 32 microglobulin (B2M) mRNA. In FIG. 1A, the relative amount of each transcript was determined using the equation 2^{-dCt}, where dCt=(Ct target gene-Ct ref gene), and Ct=cycle threshold.

Isolation of BMDMs

Bone marrow-derived macrophages (BMDMs) were generated as described previously from adult WT or MNK2-KO mice (Weischenfeldt and Porse, 2008). Briefly, bilateral femurs and tibias of mice were flushed using 26-gauge needles into sterile Hank's Balanced Salt Solution (HBSS) without Ca²⁺ and Mg²⁺ (Life Technologies). Cell clusters were disrupted by pipetting and passing the suspension through a 40 μm cell strainer. The resulting cell suspension was centrifuged (10 min, 400×g) and the cell pellet was resuspended in complete macrophage medium (CMM) containing 30% L929 cell conditioned medium (L929 cells secrete macrophage colony-stimulating factor (M-CSF) required for the promotion of bone marrow cell differentiation into macrophages), 20% fetal bovine serum (FBS), 50% DMEM (high glucose Dulbecco's Modified Eagle Medium; DMEM, Life Technologies) and with 1% (w/v) penicillin/streptomycin (Life Technologies).

Macrophage Polarisation

BMDM cells were treated for 24 h with LPS (100 ng/ml; Sigma-Aldrich, L2630-lipopolysaccharides from Escherichia coli 0111:B4) in the presence of IFNγ (20 ng/ml) to polarise towards an M1 macrophage phenotype; or cells were treated for 24 hr with IL-4 (20 ng/ml; Peprotech) alone to polarise towards an M2 phenotype.

Differentiation of 3T3-L1 Cells and Oil Red O Staining

For induction of 3T3-L1 (fibroblast) cell differentiation, pre-adipocytes were grown to 2 days post-confluence in DMEM supplemented with 10% FBS (day 0) and the medium was changed to DMEM supplemented with 10% FBS, insulin (167 nM), dexamethasone (0.5 μM), isobutylmethylxanthine (IBMX) (0.5 mM) and rosiglitazone (2 μM). After 48 h, the medium was replaced with medium containing DMEM supplemented with 10% (v/v) FBS and 167 nM insulin. On day 4, after inducing differentiation, and thereafter, the cells were cultured in DMEM with 10% FBS. The maintenance medium was changed every 48 h until the cells were utilised for experimentation (9 days from the initiation of differentiation). CGP57380 at 20 μM was added to the cells at day 0 and was maintained during subsequent media changes throughout the differentiation program.

Assessment of intracellular triglyceride levels was performed using Oil Red O staining as described by others (Smith et al., 1988). Briefly, on day 9 of differentiation of the 3T3-L1 cells were fixed in 10% formalin for 1 hour. Cells were washed in 60% isopropanol before staining in freshly prepared Oil Red O dye for 10 min. For quantification, cells were washed extensively with water to remove unbound dye, and 1 ml isopropanol was added to the stained 6-well culture plate. After 5 min, the absorbance of the oil red extract at 510 nm was measured, with an increased absorbance indicative of an increased lipid level within the cells.

Histology

Adipose tissue sections were fixed in 10% neutral buffered formalin for 6 h and dehydrated as standard before embedding in paraffin wax. Sections (4 μm) were cut and mounted on positively-charged glass slides and hematoxylin and eosin (H&E) staining was performed as standard. Slides were scanned using the Panoramic 250 Flash II scanner (3DHISTECH, Hungary). Images were analysed using Image J with the adipocyte tool macro. Adipocytes were then counted, and the absolute pixel area of each object was calculated and converted to μm².

Statistics

Analysis was performed by 2-tailed, unpaired Student's t test, one-way or two-way ANOVA as indicated in the figure legends. A P value less than 0.05 was considered to be significant. Statistical tests were performed using the statistical program GraphPad Prism (ver. 6; GraphPad Software Inc., San Diego, Calif., United States of America).

Lipolysis Assay

3T3L1 cells were differentiated as described above for 9 days. The lipolysis assay was performed according to the manufactures instructions (Abcam Lipolysis assay kit, ab185433, Abcam). Briefly, after differentiation cells were washed two times with lipolysis assay buffer. Lipolysis was stimulated using 100 nM isoproterenol for 3 h. The amount of glycerol released was measured using colorimetric intensity.

Triglyceride Measurement

The amount of tissue-released triglyceride (TAG) was measured using a Triglyceride Quantification Kit (Abcam, ab65336).

Liver Histology

Fresh frozen sections (5 μm thick) were used to detect lipid accumulation by staining with Oil Red O (Sigma). Cryosections were fixed in 60% isopropanol for 10 min and stained with 0.3% Oil Red O in 60% isopropanol for 30 min and subsequently washed with 60% isopropanol. Nuclei were stained with hematoxylin for 2 min and then rinsed with tap water. Slides were scanned using the Pannoramic 250 Flash II scanner (3DHISTECH, Hungary).

### Results and Discussion

MNK1 and 2 are Expressed in Normal Mouse Tissues Involved in Insulin-Regulated Metabolism

It has previously been shown that MNK1 and MNK2 mRNA is expressed in liver, skeletal muscle and heart. The present inventors have shown that MKNK1 and MKNK2 mRNA molecules are also expressed in adipose tissue in addition to confirming expression in the liver (see FIG. 1A). Immunoblot analysis revealed expression of the MNK1 protein in liver, skeletal and cardiac muscle and adipose tissue (FIG. 1B).

To examine the expression of MNK2 during adipocyte differentiation, 3T3-L1 fibroblasts were used, which are a widely-employed model of fat cell differentiation, to analyse the expression of standard markers such as the transcription factors PPARγ, C/EBPα and SREBP1c, and the glucose and fatty acid transporters GLUT4 and CD36, confirming the efficacy of the differentiation protocol (FIG. 1C). Levels of the MNK2 mRNA rose rapidly after induction of the differentiation protocol, with levels three-fold the levels at one day (which is at least as fast as the earliest of the other markers) (FIG. 1D). This result indicates that MNK2 plays a role early in adipocyte differentiation. The MNK2 mRNA levels fell to approximately pre-differentiation levels by day 6. In contrast, MKNK1 mRNA levels increased only slightly over this period. In further work (see FIG. 1E), it was found that MNK2 but not MNK1 is upregulated very early on during the differentiation of 3T3-L1 pre-adipocytes into adipocytes, before the appearance of most of the key genes involved in adipogenesis (apart from C/EBPβ).

MNK2-KO Mice are Protected Against HFD-Induced Fat Gain and Indices of Insulin Resistance

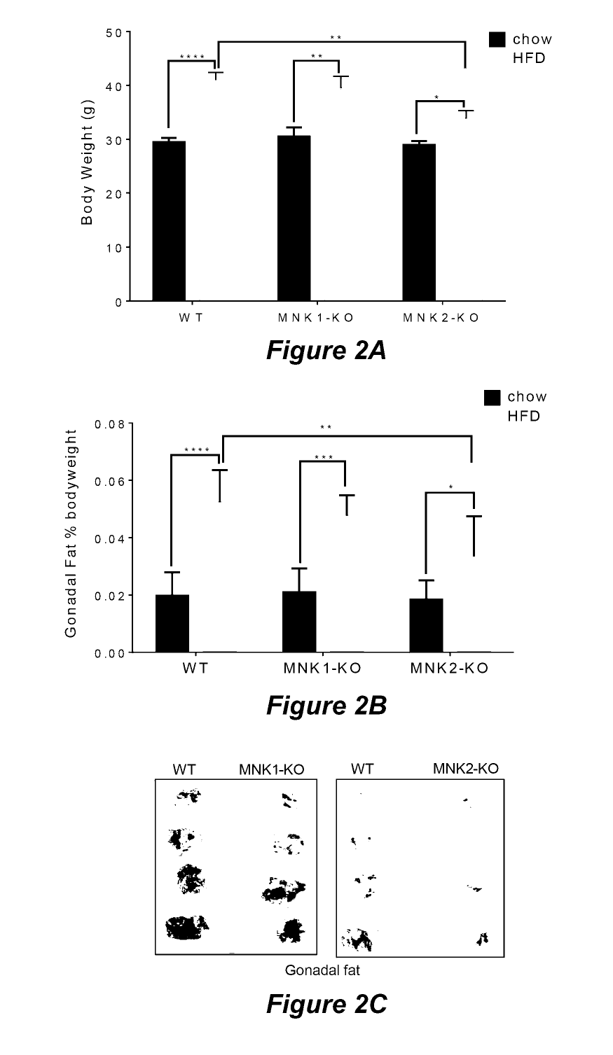

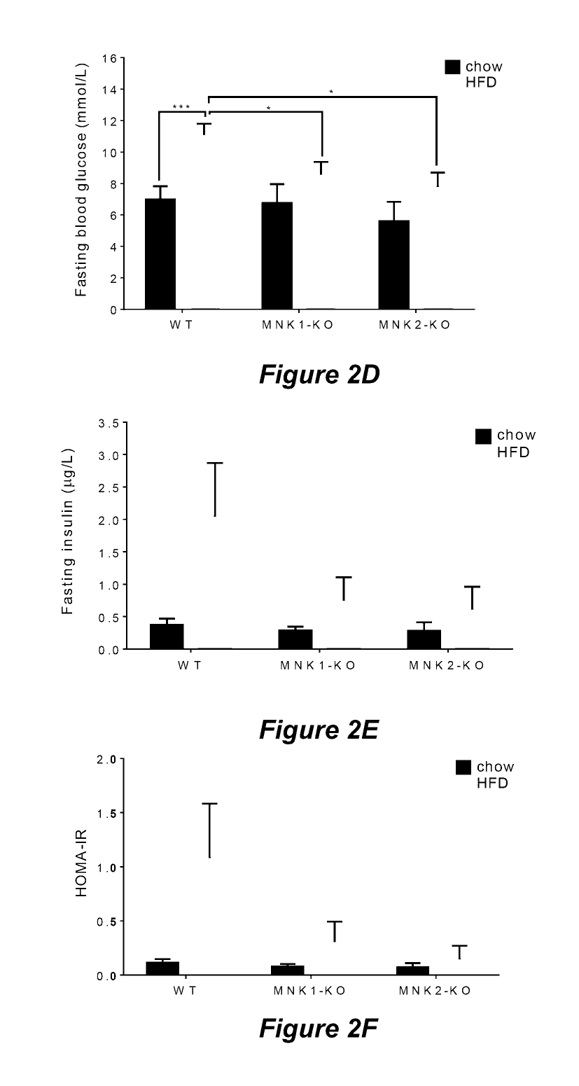

The response of MNK1-knockout (KO) or MNK2-KO mice, as compared to wild-type (WT) mice, to a high fat diet (HFD) as compared to a normal diet (chow) was investigated to determine the roles of MNK-1 and MNK-2. Feeding wild-type (WT) C57B16/J mice a HFD compared to a chow diet led to increased bodyweight and gonadal fat (FIG. 2A-2C). High fat-fed MNK1-KO mice showed similar increases in body and gonadal fat weight to WT mice on the HFD (FIG. 2A-2C). In contrast, feeding homozygous MNK2-KO mice the same HFD caused smaller increases in body weight and gonadal fat. In WT mice, the HFD also caused marked increases in circulating levels of glucose and insulin (FIG. 2D, 2E, respectively). Such increases were markedly reduced in both MNK1-KO and MNK2-KO animals on the HFD, indicating attenuation of the adverse effects of the HFD. Notably, on the chow diet, the MNK1-KO and MNK2-KO mice had similar body weight, gonadal fat weight, basal glucose and insulin levels to those of WT mice. No difference in food intake of MNK-KO mice compared to wild-type controls was observed.

The homeostatic model assessment of insulin resistance (HOMA-IR) is widely-used as an index of insulin resistance; it is calculated from blood insulin and glucose levels (Matthews et al. 1985). The HOMA-IR was elevated in WT/HFD mice (FIG. 2F). However, in MNK2-KO/HFD mice, much less increase in HOMA-IR was observed compared to chow fed animals, indicating MNK2-KO animals are largely protected from the adverse effects of the HFD such as insulin resistance. Although MNK1-KO mice show similar weight and fat gain to WT mice on the HFD, they also displayed lower blood insulin and glucose levels and thus a better HOMA-IR than WT/HFD mice (FIG. 2D-2F).

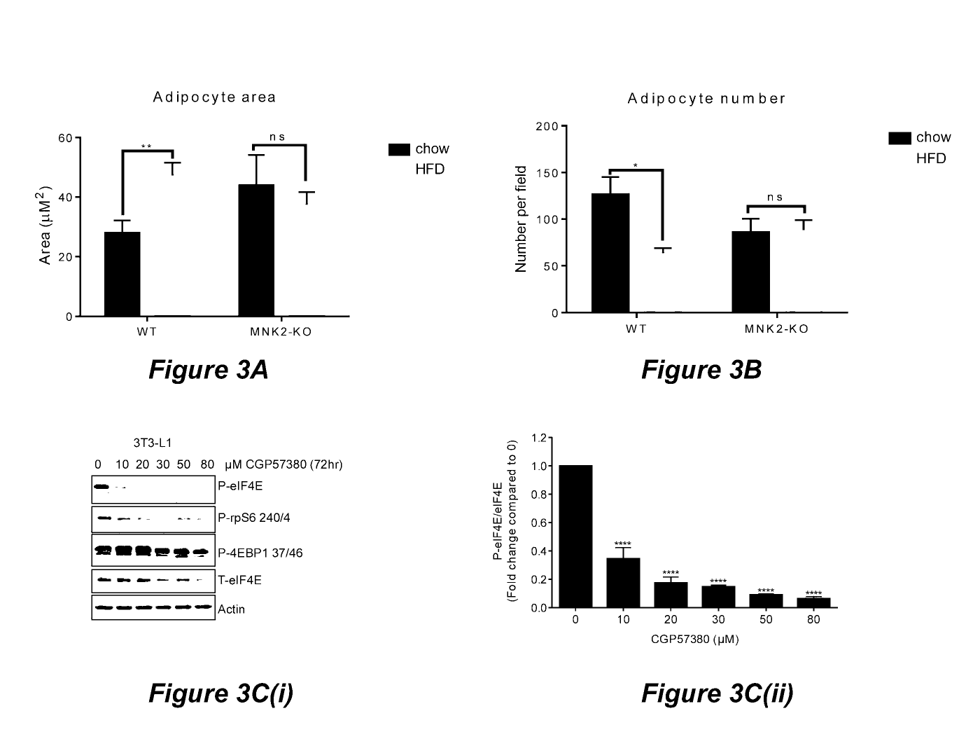

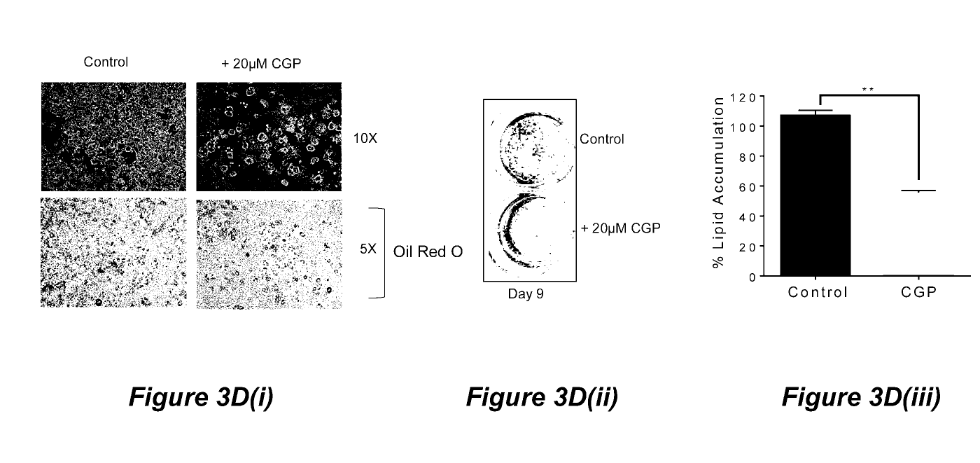

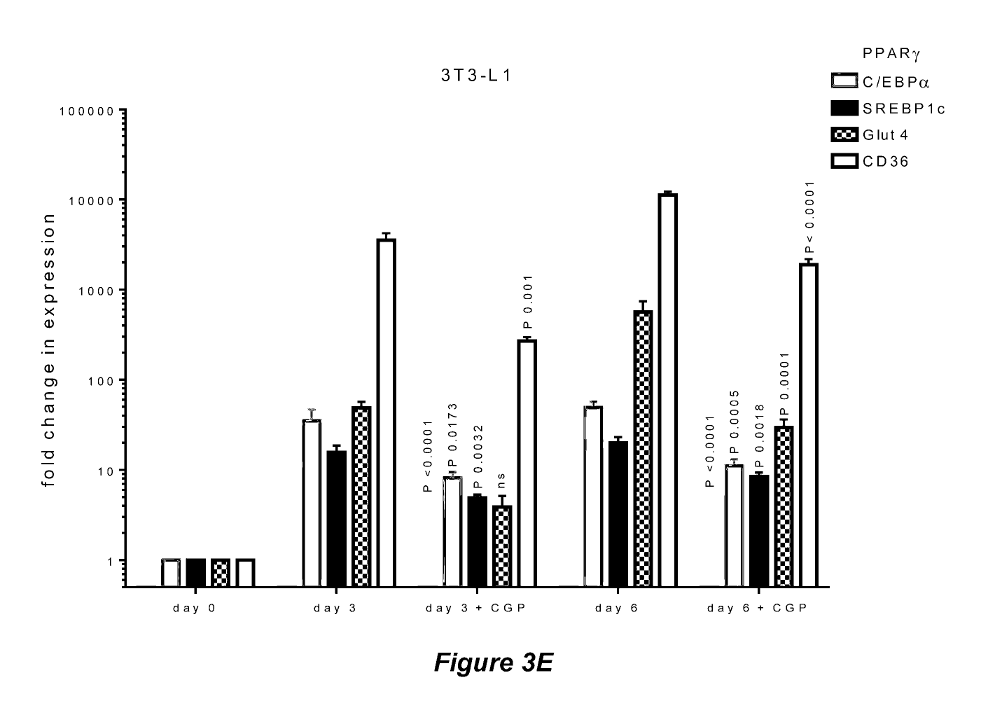

In WT mice on a HFD, adipocyte size increased substantially compared to those on the chow diet, as assessed by cell area (1.7 fold; FIG. 3A). Assuming these cells are roughly spherical, this amounts to an approximately 3-fold increase in adipocyte volume. Consistent with this, there was a corresponding decrease in the observed number of adipocytes for HFD-fed WT as compared to chow fed mice (FIG. 3B). In contrast, there was no increase in size of adipocytes in MNK2-KO/HFD mice when compared to chow-fed controls (FIG. 3A, 3B). This suggests a possible defect in adipocyte lipid storage when these animals are placed on the HFD. Interestingly, adipocyte size was found to be larger in MNK2/chow animals than WT controls, and while not wishing to be bound by theory, this may indicate that there is a deficit in adipogenesis in MNK2-KO mice, so there are fewer adipocytes, and each consequently becomes bigger. The observation that adipocyte size did not increase on the HFD in MNK2-KO mice likely contributes to the reduced weight gain of high fat-fed MNK2-KO mice.

MNKs are Required for Adipocyte Differentiation

The reduced increase in fat tissue observed in the MNK2-KO/HFD mice compared to WT/HFD controls and the increased expression of MNK2 during adipocytic differentiation (FIG. 1C) prompted an examination of whether MNK2 plays a role in the differentiation of cells into adipocytes. To this end, 3T3-L1 cells were differentiated into adipocytes using a standard protocol and differentiation was monitored by assessing lipid content and evaluating adipocyte markers such as the mRNAs for PPARγ, C/EBPα, SREBP1c, GLUT4 and CD36.

The effect of CGP57380, a widely-used inhibitor of the activities of both MNK1 and MNK2 (Tschopp et al., 2000), on the differentiation of 3T3-L1 cells was investigated. A dose-response study was conducted to determine the concentration of CGP57380 required to block MNK function, as assessed using phosphorylation of eukaryotic translation initiation factor 4E (eIF4E) as a read-out. MNKs are the only kinases known to phosphorylate eIF4E, and accordingly, the phosphorylation of eIF4E (P-eIF4E) provides a direct read-out of MNK biological activity. The results showed that IM CGP57380 was effective, almost completely blocking MNK activity in 3T3-L1 cells (FIG. 3C). Further, as shown in FIG. 3D, 20 μM CGP57380 inhibited the accumulation of lipid into 3T3-L1 cells subjected to the differentiation protocol.

To study whether the inhibition of lipid accumulation reflected altered expression of other genes involved in adipogenic differentiation, the mRNA levels for PPARγ, C/EBPα, SREBP1c, GLUT4 and CD36 were examined. As shown in FIG. 3E, CGP57380 substantially blocked the induction of all of these genes at both day 3 and 6 of the differentiation protocol compared to 3T3-L1 cells differentiated in the absence of CGP57380. These data indicate that MNKs play a role in the differentiation of 3T3-L1 cells into adipocytes, and that they may also play a role in adipocyte differentiation in vivo, and confirms the role of the MNK for adipocyte size and number shown in FIGS. 3A and 3B.

MNK2-KO/HFD Mice do not Show Increased Liver Lipid Accumulation

Given the attenuated gain in adipose tissue observed in MNK2-KO mice compared to WT animals on the HFD, an investigation was conducted to assess whether the extra lipid load from the diet might be being diverted to the liver and contribute to fat accumulation there. HFD MNK2-KO mice showed similar liver weights to WT mice on the HFD (data not shown). Total liver lipid levels of WT and MNK2-KO mice on the HFD were similar (data not shown). These data indicate that the extra lipid in the HFD is not being re-directed to the liver in MNK2-KO mice.

MNK-KO Mice Exhibit Improved Glucose Tolerance Compared to WT Mice on the HFD

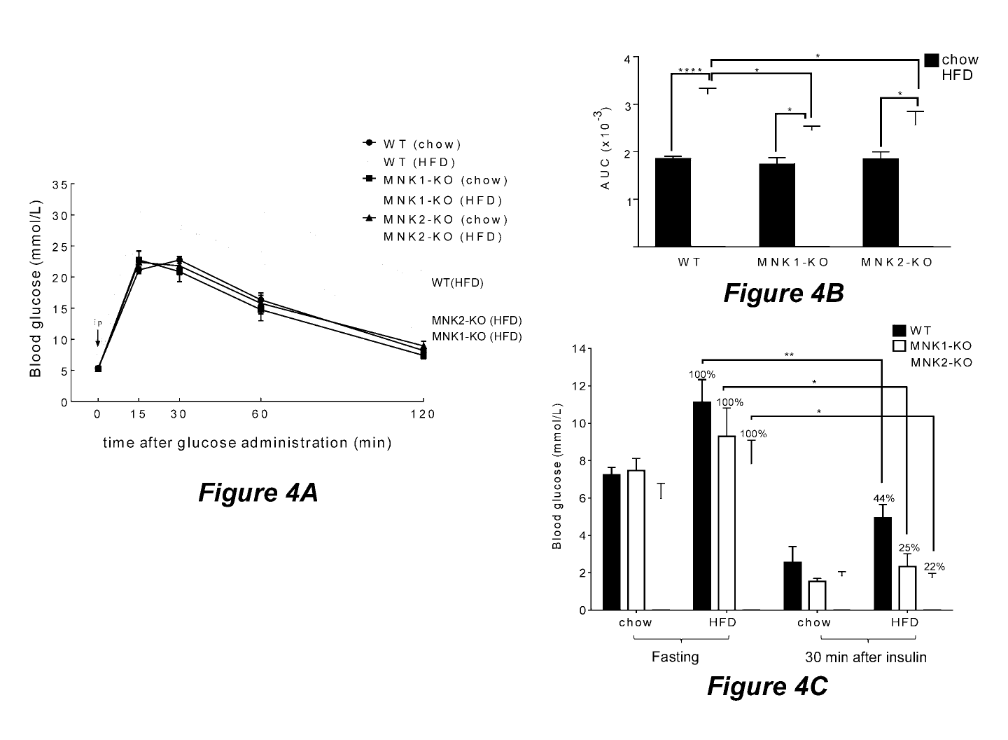

To assess directly the effect of the HFD on glucose tolerance in WT and MNK2-KO animals, a glucose tolerance test (GTT) was performed. Chow-fed MNK1-KO or MNK2-KO mice showed a similar response to chow-fed WT animals in the GTT indicating that the MNKs do not affect glucose tolerance under normal (chow) conditions. However, MNK1-KO and MNK2-KO/HFD mice showed markedly lower blood glucose levels at all times after glucose administration relative to WT/HFD mice, indicating a role for MNK1 and MNK2 in regulating glucose handling (FIG. 4A, 4B), and accordingly, higher glucose tolerance. Thus, from these results, the knock-out of either MNK1 or MNK2 appears to protect mice against HFD-induced glucose intolerance.

In order to assess the action of insulin, WT, MNK1-KO and MNK2-KO mice on chow or the HFD were treated with insulin to assess the ability of these animals to decrease blood glucose levels. Insulin decreased blood glucose more effectively in MNK1-KO or MNK2-KO/HFD animals than in WT/HFD mice, indicating that insulin acts more efficiently in MNK1-KO/HFD and MNK2-KO/HFD mice (FIG. 4C). Indeed, the percentage reduction in blood glucose levels caused by insulin in MNK1-KO or MNK2-KO/HFD mice is similar to that observed in animals fed the chow diet. These data indicate that MNK1-KO and MNK2-KO mice are each protected to a considerable extent against HFD-induced insulin resistance.

MNK2-KO Mice Show Better Insulin Signalling than WT Mice on the HFD

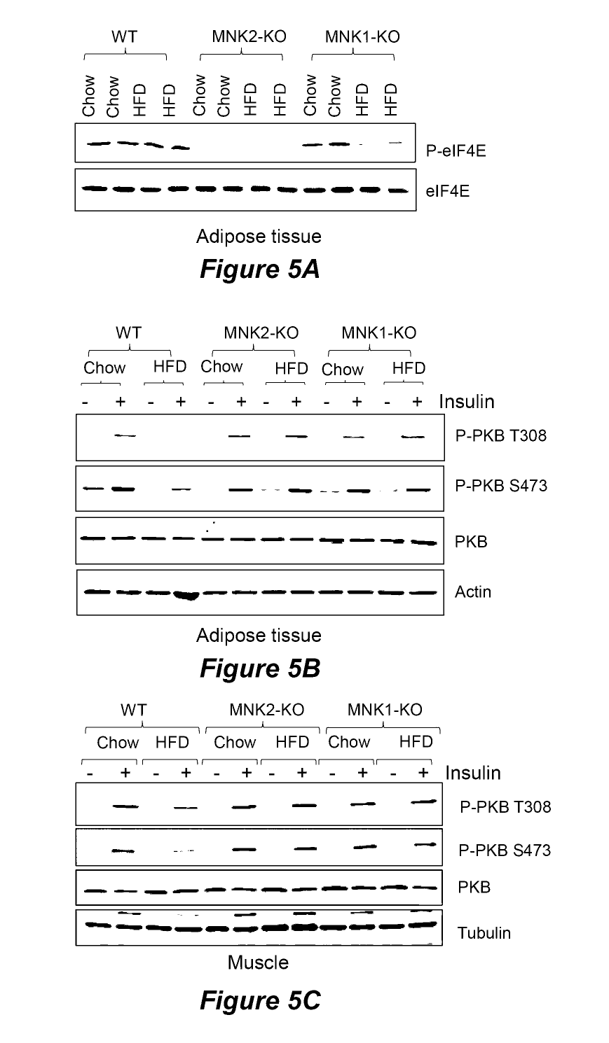

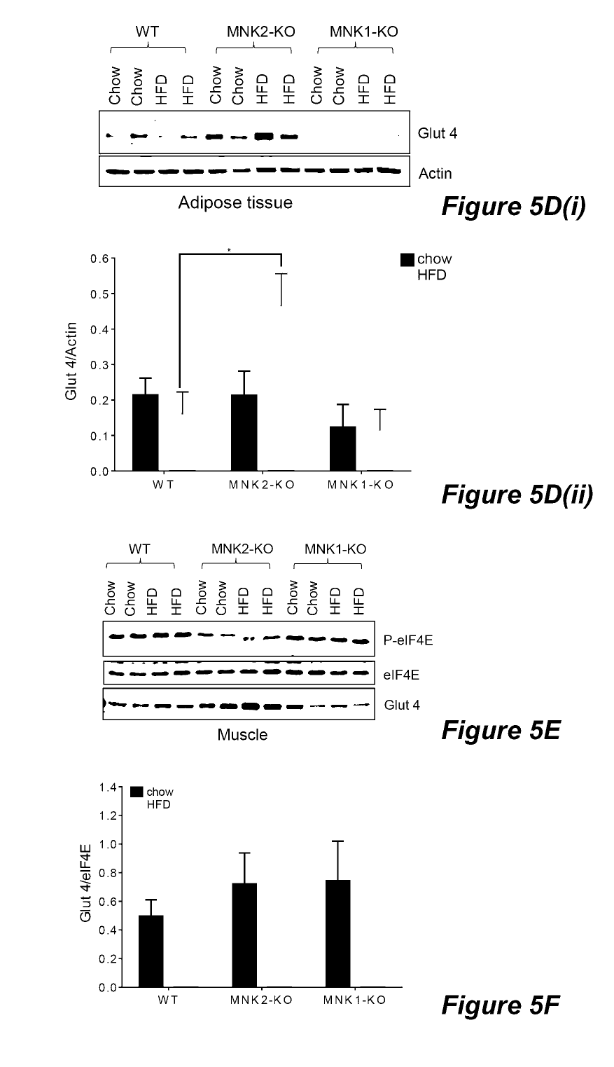

To assess the relative levels of MNK1 and MNK2 activity in adipose tissue, the phosphorylation of eIF4E (the common substrate of MNK1 and MNK2) was examined. The HFD caused a small increase in phosphorylated (P)-eIF4E (FIG. 5A) in the adipose tissue of WT mice. MNK2-KO mice showed a substantial decrease in P-eIF4E under both dietary conditions, whereas MNK1-KO mice showed no change in P-eIF4E on the chow diet compared to WT animals on the chow diet. This demonstrates that MNK2 is the most active MNK isoform in adipose tissue. In MNK1-KO mice, P-eIF4E was lower in adipose tissue of HFD-fed than chow-fed mice (FIG. 5A).

Insulin stimulates the uptake of glucose into tissues such as fat and especially muscle through the translocation of the glucose transporter GLUT4 to the plasma membrane. This effect, like many of the metabolic effects of insulin, is mediated through protein kinase B (PKB, also termed Akt) which is phosphorylated and activated downstream of phosphatidylinositide 3-kinase (PI 3-kinase). To assess the ability of insulin to activate this pathway, WT and MNK2-KO mice, fed either chow or the HFD, were administered insulin and sacrificed 30 min later to test for insulin resistance. Samples were taken of blood and also of adipose tissue and skeletal (gastrocnemius) muscle. Tissue samples were analysed by immunoblot for various parameters of insulin signalling. In WT mice, insulin administration caused an increase in the phosphorylation of PKB at Ser473 and Thr308, the main site involved in its activation in adipose tissue (FIG. 5B) and in muscle (FIG. 5B, 5C). These effects were reduced in WT animals fed the HFD indicating partial resistance to the effects of insulin. In MNK1-KO or MNK2-KO mice on the chow diet, insulin-induced PKB phosphorylation was similar to WT animals on the chow diet, but insulin-induced PKB phosphorylation was higher in MNK1-KO or MNK2-KO mice on the HFD than in WT/HFD animals, showing that insulin resistance is attenuated in both of the MNK-KO/HFD mice (FIG. 5B, 5C).

An increase in total GLUT4 protein was observed in the adipose tissue of MNK2-KO/HFD mice compared either to WT or chow-fed MNK2-KO animals (FIG. 5D). GLUT4 is the key insulin-regulated glucose transporter, which mediates uptake of glucose into insulin-responsive tissues in response to this hormone. Insulin promotes its translocation to the plasma membrane through a signalling pathway involving PKB. Thus, the improved glucose tolerance of MNK2 KO/HFD animals compared to WT/HFD mice likely involves a combination of improved insulin sensitivity and/or signalling and higher levels of GLUT4 protein. Glut4 mRNA levels were slightly lower in adipose tissue of MNK2-KO animals on chow or the HFD compared to the WT animals (data not shown). GLUT4 protein levels tended to be lower in fat of MNK1-KO animals compared to wild type animals (FIG. 5D). In skeletal muscle, eIF4E phosphorylation was decreased in MNK2-KO mice, but not in MNK1-KO animals (FIG. 5E), indicating that MNK2 is the most active MNK isoform in this tissue but that MNK1 also contributes. Insulin-induced PKB phosphorylation was reduced in WT mice fed the HFD compared to the chow diet, whereas it was not impaired in MNK1-KO or MNK2-KO animals. GLUT4 protein levels tended to be higher in muscle of MNK2-KO mice, especially those on the HFD, but the difference did not reach significance (FIG. 5E-5F). Thus, MNK1 and MNK2 both play roles in impairing insulin signalling in adipose tissue and muscle of HFD-fed mice.

MNK2-KO Mice are Protected Against HFD-Induced Adipose Inflammation

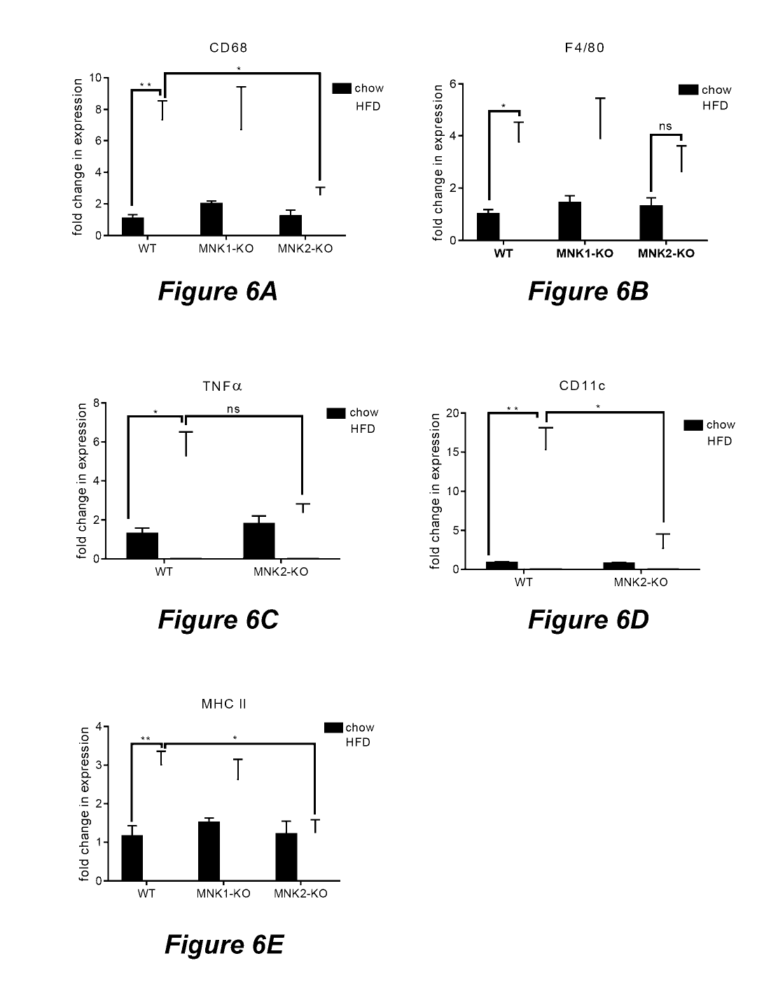

A consequence of consuming a HFD, in addition to weight gain and insulin resistance, is inflammation, especially in adipose tissue, which becomes infiltrated with pro-inflammatory M1 macrophages. Markers for macrophages and M1-polarised macrophages in adipose tissue from WT, MNK1-KO and MNK2-KO mice, which had been fed either chow or the HFD, were examined. WT/HFD and MNK1-KO/HFD mice showed increases, compared to WT/HFD animals, in mRNA levels for macrophage markers such as Cd68 and F4/80 (FIG. 6A, 6B). In view of the altered macrophage markers in MNK2-KO/HFD mice, the pro-inflammatory (M1 macrophage) markers Cd11c and Tnfα (FIG. 6C, 6D), the chemokine receptors Ccr2 and Ccr5 (important in macrophage trafficking; data not shown), and Mmp12 (matrix metalloproteinase 12; data not shown) and Adam8 (data not shown), which are involved in digesting extracellular matrix to allow cell migration. In marked contrast, MNK2-KO/HFD mice, but not MNK1-KO/HFD mice, showed sharply reduced inflammation compared to the same animals on chow diets, with less or no increase in Cd68, F4/80, Tnfa Cd11c, Mhc II mRNAs (FIGS. 6A-6D), as well as Ccr2, Ccr5, Mmp12 or Adam8 mRNAs (data not shown). Taken together, these data show that, on the HFD, the adipose tissue of MNK2-KO mice does not become heavily infiltrated with macrophages (based on the absence of the normal marked increase in Cd68 and F4/80 mRNAs) or show an increase in pro-inflammatory M1 macrophages, as indicated by the marked blunting of the levels of Cd11c and Tnfc. The lack of increase in MhcII in the MNK2-KO mice is of interest, as adipocyte MHCII is reported to play an important role in attracting immune cells to adipose tissue in HFD-fed animals. Further, a diet high in saturated fats has been reported to cause insulin resistance via the activation of c-jun amino terminal kinase (JNK). However, the MNKs are not activated by JNK ruling out a role for JNK in the phenotype observed in HFD-MNK2-KO mice (Waskiewicz et al., 1997).

Accordingly, the results show that the HFD triggers extensive inflammation in adipose tissue of WT mice, while MNK2-KO mice are protected against the pro-inflammatory effects of the HFD. Interestingly, although MNK1-KO/HFD mice show partial protection against insulin resistance and glucose intolerance, they still exhibit similar adipose tissue inflammation to WT/HFD animals. This underscores the distinct roles of MNK1 and MNK2 in responses to high fat feeding. However, it is striking that the basis of the protection appears to differ; MNK2-KO mice show reduced fat gain and inflammation alongside their protection whereas MNK1-KO mice increase weight and fat tissue similarly to WT mice, but are nonetheless protected against insulin resistance.

Macrophage Biology

Macrophages from MNK2-KO mice were assessed to determine whether they were intrinsically defective in producing cytokines such as TNFα and IL-6. Both MNK1 and MNK2 contribute to eIF4E phosphorylation in bone marrow-derived macrophages (BMDMs) (data not shown). Consistent with their regulatory characteristics, the increase in elF4E induced by lipopolysaccharide (LPS) in WT BMDMs was lost in MNK1-KO cells (data not shown). BMDMs from wild-type or MNK2-KO mice were stimulated in vitro with lipopolysaccharide (LPS), and cytokine mRNA levels were assessed. LPS increased the levels of the Tnfα and Il-6 mRNAs in wild-type and MNK2-KO BMDMs to similar extents (data not shown). These data indicate there is no intrinsic defect in the ability of MNK2-KO BMDMs to respond to LPS and produce cytokines.

Longer term (24 h) effects of a combination of LPS and IFNγ were examined to assess the polarisation of BMDMs towards the pro-inflammatory M1 phenotype. WT and MNK2-KO BMDMs responded similarly, indicating that MNK2 is not required for the production of these pro-inflammatory cytokines by BMDMs (data not shown).

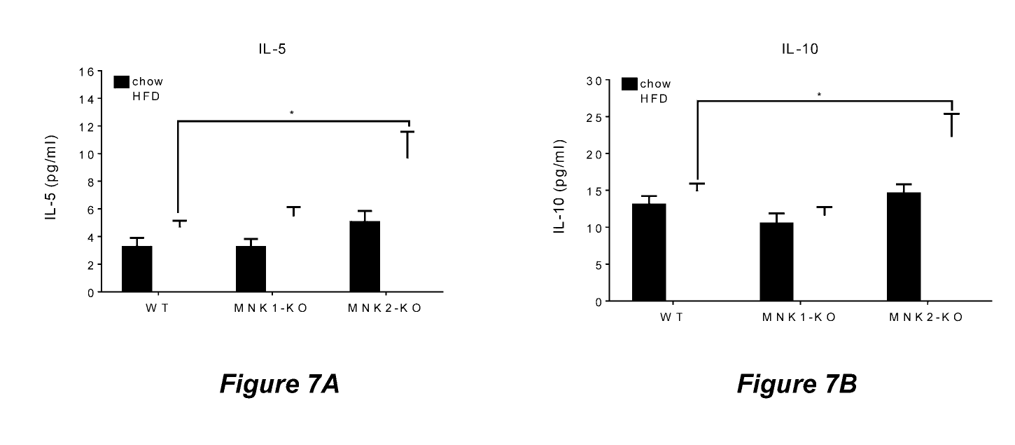

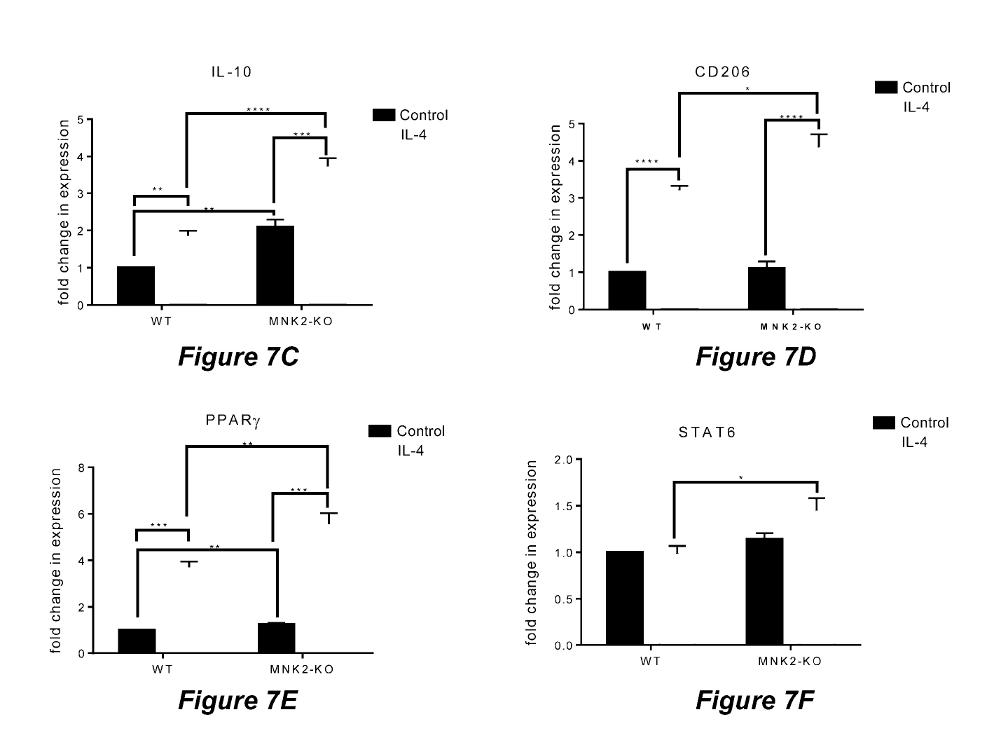

In the plasma of the MNK2-KO/HFD mice, a significant increase in the mRNA levels of two anti-inflammatory markers, Il-10 and Il-5, was observed compared to WT/HFD mice (FIG. 7A, 7B). The intrinsic abilities of BMDMs from WT and MNK2-KO mice to polarise towards an M2 anti-inflammatory phenotype were investigated. Interestingly, it was found that BMDMs from MNK2-KO mice had elevated levels of Il-10 and Ppary under control conditions compared to WT BMDM and that all markers of M2 polarisation examined were increased after 24 h stimulation with IL-4 (FIG. 7C-7F). Data for phosphorylated eIF4E indicate that MNK2 is the main isoform in macrophages (data not shown). Taken together, the data suggest that macrophages from MNK2-KO mice have a higher tendency towards an anti-inflammatory phenotype.

High Fat Feeding Induces Nrf2 Expression in MNK2-KO but not Wild-Type Mice

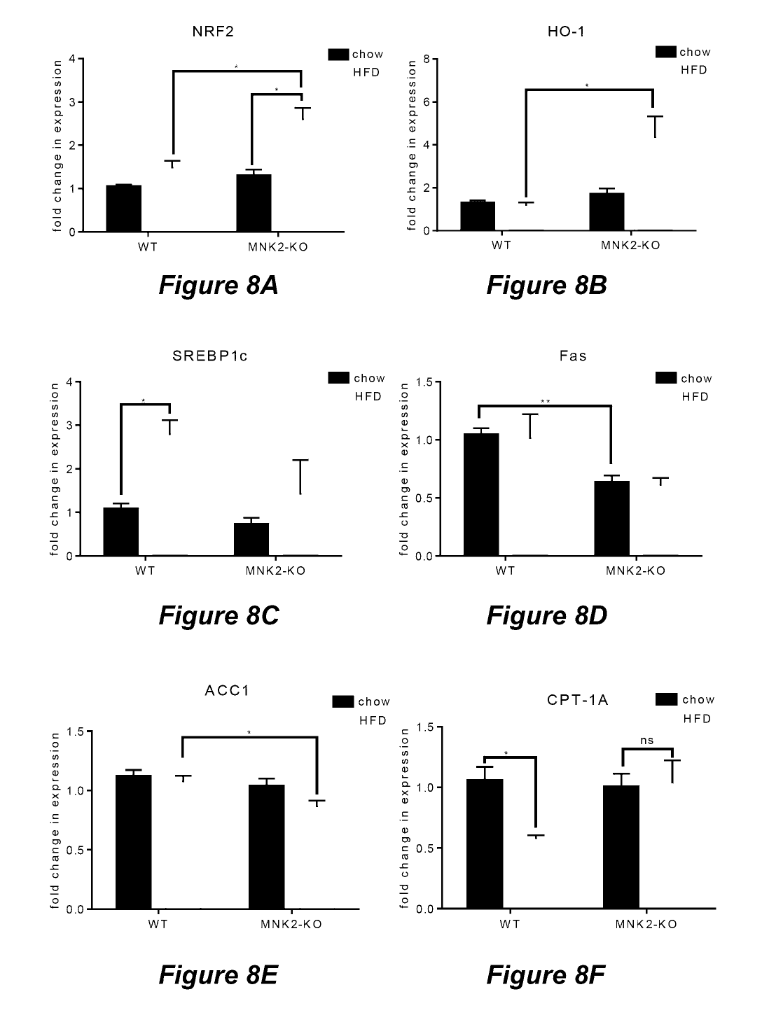

Nuclear factor erythroid 2-related factor 2 (Nrf2) plays an important role in protection against oxidative stress, which can arise from extensive fatty acid oxidation, and is also reported to protect against the development of metabolic syndrome. Given that MNK2-KO mice show protection against indices of metabolic syndrome, the expression of Nrf2 in the livers of WT and MNK2-KO mice was examined. Levels were similar in mice fed a chow diet. Nrf2 mRNA levels increased markedly in MNK2-KO mice fed a HFD, but not in the corresponding control animals (FIG. 8A). Hepatic Nrf2 protein levels also showed an increase in MNK2-KO mice, but the changes did not reach significance. Therefore, to confirm increased Nrf2 function, the expression of a well-characterised Nrf2 target gene, haem oxygenase-1 (HO-1), was also tested, which showed a similar pattern to the protein expression of Nrf2 itself (FIG. 8B). Interestingly, activation of Nrf2 is reported to protect against steatosis. Nrf2 can be transcriptionally controlled by PPARα; however, no change in PPARα levels was observed in liver of MNK2-KO or WT mice on the HFD (data not shown).

Treating Palmitate-Induced Insulin Resistance

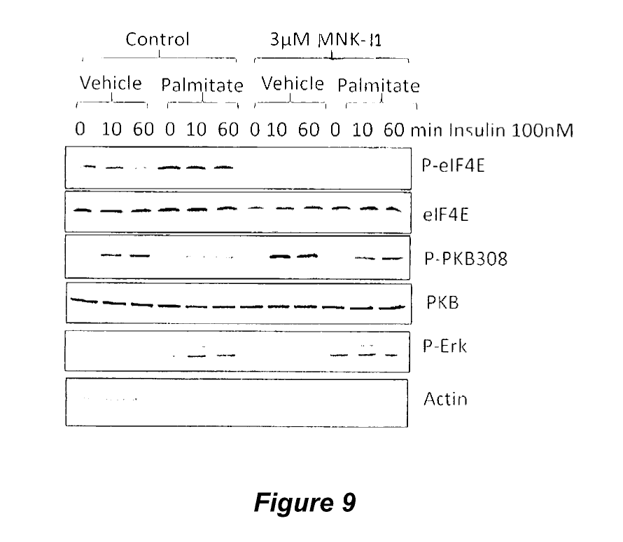

Using a C2C12 skeletal muscle model of insulin resistance, it was observed that palmitate (a free fatty acid whose levels increase in blood on a high fat diet) treatment increases P-eIF4E; thereby indicating that palmitate activates the MNK enzymes. Further experimentation was conducted using the C2C12 cells treated for 16 hours with 4 mM palmitate prior to stimulation with 100 nM insulin for 10 and 60 minutes. Some of the cells were further treated with 3 μM of the MNK inhibitor, Mnk-I1. The results (see FIG. 9) showed that the inhibitor inactivated MNK1 and MNK2 (see reduced levels of P-eIF4E in the treated cells), but the insulin signalling marker P-PKB308 showed that the MNK inhibitor is able to restore insulin signalling during palmitate exposure.

### Example 2 Generation of MNK1 and MNK2 Double Knockout Animals and Studies with a High Fat Diet (HFD)

Studies in WT and MNK-KO Mice

The studies described in this example were performed using mice as described in Example 1 back-crossed onto stock C57B1/6 mice for 5-6 generations. Heterozygous MNK1-KO (Mknk1^{+/-}) and MNK2-KO (Mknk2^{+/-}) mice were then crossed to obtain WT and MNK1+MNK2 double KO (Mknk1^{-/-}; Mknk2^{-/-}; DKO)) animals, as well as MNK1-KO (Mknk1^{-/-}) and MNK-KO (Mkn2^{-/-}) animals. It had previously been reported that knockout of MNK1 and MNK2 does not affect the development, viability or fertility of mice (Ueda et al., 2004) and consistent with this, no adverse effects of the knockout of MNK1 plus MNK2 were observed in mice fed either the chow or high-fat diets. The chow diet and HFD were similar to that described in Example 1. In this case, the chow diet formulation was the Teklad Global 18% Protein Rodent Diet (Envigo, Madison, Wis., United States of America) and the HFD formulation was sourced from Specialty Feeds Pty Ltd (Diet SF15-095; Glen Forrest, Wash., Australia).

Weight Gain on a HFD

On a chow diet, WT and DKO mice gained weight to similar extents over the 16-week period of the study (data not shown), although at 4 weeks of age, MNK1-KO were slightly heavier than MNK2-KO or WT mice (data not shown). As GTTs were performed in week 15-16, the data obtained after that time point are not completely reliable due to the disturbance encountered by the mice.

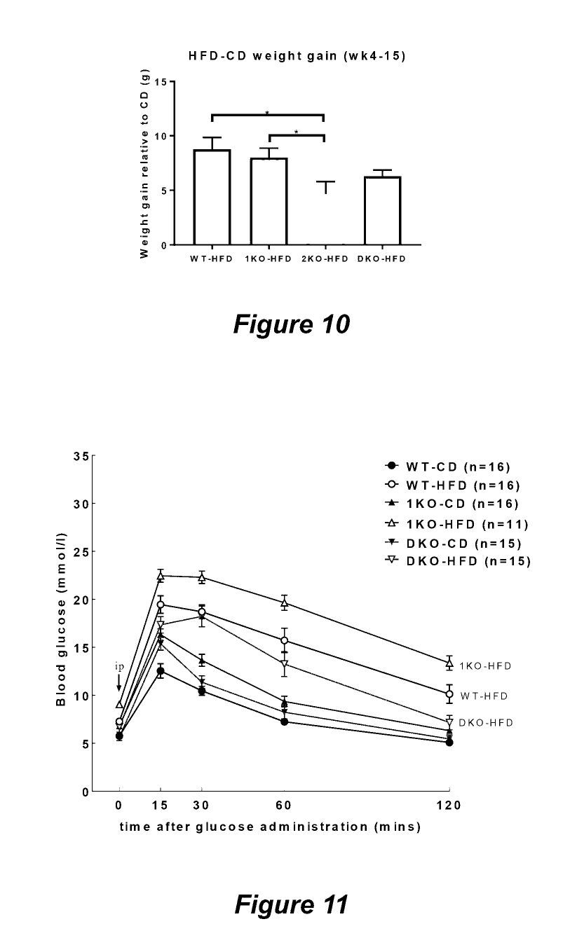

To test the animals' response to calorie-overload, the mice were offered chow (control) or an energy-rich high fat diet (HFD) from weaning (ie from 4 weeks of age), for a further 16 weeks (until age 20). On the HFD, WT mice gained considerably more weight (25 or 28 g, at 11 weeks or 15 weeks on HFD). Importantly, and consistent with observations described in Example 1, MNK2-KO mice gain substantially less weight on the HFD than WT controls (FIG. 10) confirming that loss of MNK2 protects against weight gain on a HFD. However, in contrast to the results described in Example 1, the MNK1-KO mice in this case actually gained weight similarly to WT controls (FIG. 10); the reason(s) for this difference may lie in the fact these animals are not genetically identical to those used earlier, differences in the HFD used, or other factors. With the DKO mice, it was found that the animals show slightly greater weight gain than MNK2-KO mice by 15 weeks, although the gain is still rather less than that seen for WT or MNK1-KO animals (FIG. 10). This was unexpected as it had been speculated that the knockout of both MNKs would offer greater protection against weight gain than KO of each MNK isoform alone, which offers partial protection. The data therefore show that disabling MNK1 and MNK2 does not offer greater benefit than loss of MNK2 alone, and may actually offer less advantage in preventing weight gain.

GTT Data

Glucose tolerance tests were performed as described in Example 1 to assess whether the HFD-fed animals developed glucose intolerance (which is generally caused by insulin resistance in this setting). After 11 weeks on either the chow or HFD, a glucose tolerance test (GTT) was performed after 6 hours of fasting. Fasting glucose concentration was measured from the blood bleeding from tail tip before the mice were intra-peritoneally (ip) injected with 25% D-glucose solution (2 g/kg body weight; Sigma-Adrich, Australia), and blood glucose concentrations were measured at 15, 30, 60 and 120 min post ip injection using a Freestyle Lite glucometer (Abbott, Macquarie Park, NSW,Australia). Mice fed a HFD show impaired glucose tolerance. Given that the study described in Example 1 showed that HFD-fed MNK1-KO or MNK2-KO mice show better glucose tolerance than corresponding WT animals, it was anticipated that MNK1+2-DKO mice would show even better glucose tolerance. However, as shown in FIG. 11, this is not the case. Indeed, it was found that MNK1-KO mice do not show better glucose tolerance than WT-HFD mice. Further, following a bolus of glucose, in HFD-fed MNK-DKO mice, plasma glucose levels return almost to baseline by 120 min, similar to the situation for chow-fed mice of any of these genotypes. Thus, in common with MNK2-KO mice (see Example 1), DKO animals show better glucose tolerance than WT mice fed an HFD, as was seen by computing the area under the curve (AUC) for these data (data not shown). Assessment of the AUC also revealed that MNK1-KO/HFD mice actually showed a worse glucose tolerance than WT/HFD animals and much worse than MNK-DKO/HFD mice, with glucose concentrations remaining markedly higher in MNK1-KO/HFD mice (FIG. 11).

Thus, loss of MNK1 does not appear to consistently protect against HFD-induced glucose intolerance. This may help to explain why loss of MNK1 and MNK2 does not give further benefit over loss of MNK2 alone in respect of their performance in the GTT of HFD-fed mice.

Taken together, the data provided in this example indicate that knocking out MNK2 is consistently beneficial in terms of improved glucose tolerance (metabolic health) on a HFD. In contrast, the effects of loss of MNK1 appear to vary, probably dependent on the exact genetic background and/or the precise components of the diet. Further, knockout of both MNKs does not offer a superior effect to the loss of MNK2 alone. Accordingly, in a method of treating a pre-diabetic in accordance with the present disclosure, it will be much more reliable and effective to use specific inhibitors of MNK2 rather than dual inhibitors that affect both enzymes.

### Example 3 the Effects of MNK2-Specific Inhibition on eIF4E Phosphorylation in 3T3-L1 Cells

Studies in 3T3-L1 Cells

As described in Example 1, it was found that MNK2-KO mice on a normal chow diet contain the same amount of gonadal fat tissue, but that such tissue has fewer, larger fat cells (adipocytes). This suggests a defect in the production of fat cells (ie adipocyte differentiation). On a HFD, fat cells usually become larger, allowing animals to store more fat and become heavier. However, in the MNK2-KO mice, the adipocytes did not become larger on the HFD, likely explaining why these mice do not become as heavy as WT/HFD mice. While not wishing to be bound by theory, this lack of further size increase could reflect (i) the fact that the larger fat cells in MNK2-KO mice have reached storage capacity and cannot increase their fat content and/or (ii) that the intrinsic ability of such cells to store fat is impaired.

In order to study the role of MNK2 in adipocyte differentiation, 3T3-L1 cells were used in a similar manner to that described in Example 1. 3T3-L1 fibroblasts differentiate into adipocytes when incubated with a combination of isobutylmethylxanthine (IBMX, 500 μM; raises cAMP), insulin (350 nM), dexamethasone (0.5 μM; a steroid) and rosiglitazone (2 μM; stimulates the transcription factor PPARγ). In response to this “cocktail” of stimuli, these cells first undergo clonal expansion followed by induction of a cascade of transcription factors resulting in the expression of genes involved in adipogenesis:

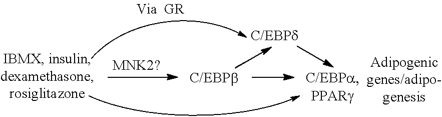

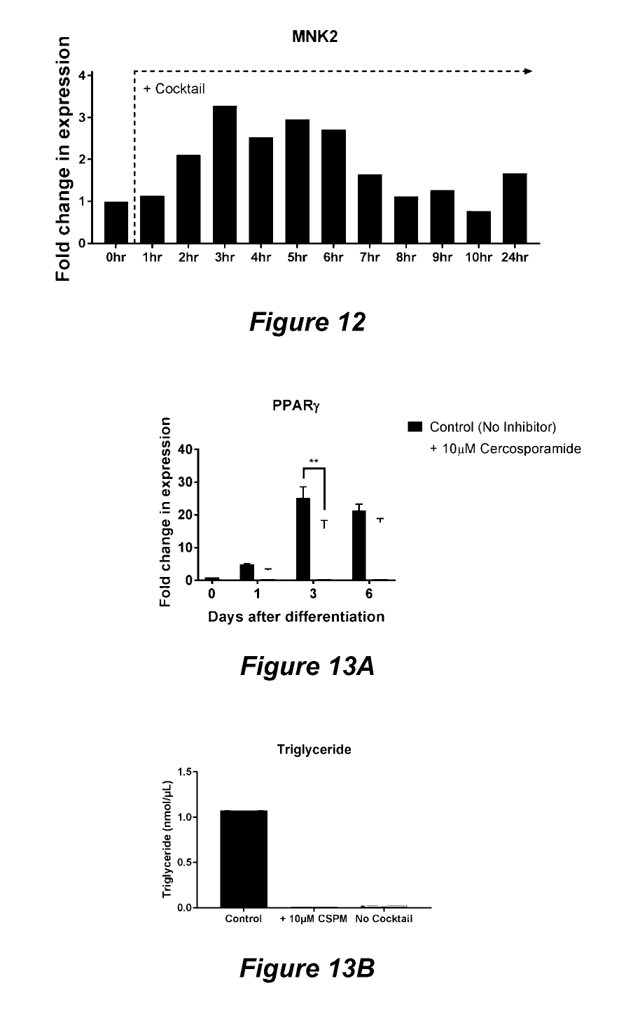

The results (shown in FIG. 12) revealed that the Mnk2 mRNA is induced very rapidly after the addition of the differentiation cocktail (>3-fold by 3 h, which is sustained until around 6 h, after which Mnk2 mRNA expression declines), positioning it to play a role in adipogenesis. In Example 1 it was shown that the MNK inhibitor CGP57380 impairs the induction of key transcription factors such as C/EBPβ, PPARγ and SREBP1c (which drives expression of genes involved in lipid storage). It was also found that CGP57380 inhibits the expression of the glucose and lipid transporters GLUT4 and CD36, which may at least partly explain why fat storage is restricted in MNK2-KO adipocytes in vivo. Here, studies were undertaken to test another, quite distinct compound that also inhibits the MNKs, namely cercosporamide. As shown in FIG. 13, cercosporamide impaired the induction of genes such as PPARγ (FIG. 13A) and also reduced lipid accumulation (FIG. 13B).

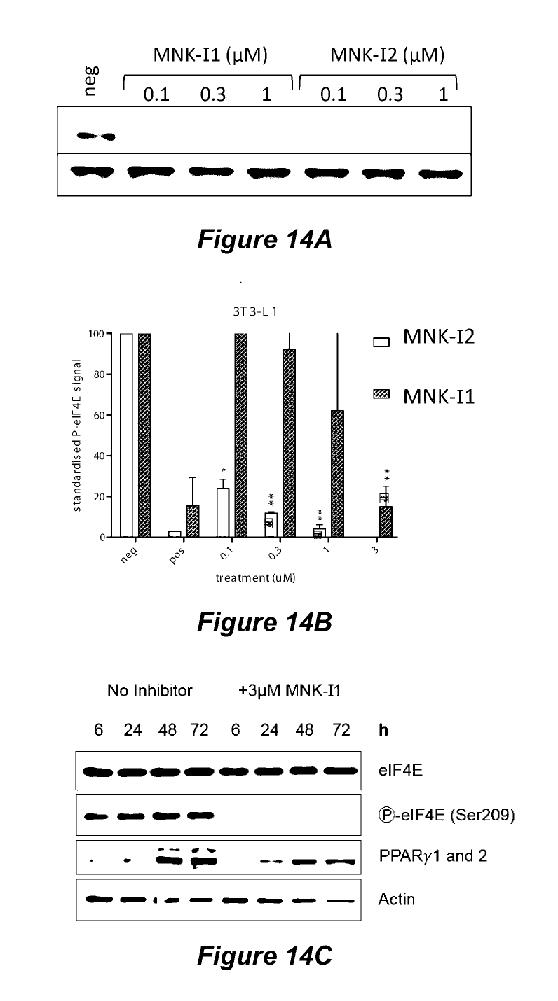

However, CGP57380 and cercosporamide do inhibit other protein kinases in addition to the MNKs and are not very potent MNK inhibitors (Bain et al., 2007; Konicek et al., 2011); for instance, it has to be used at concentrations ≥20 μM to strongly inhibit P-eIF4E. Consequently, a further inhibitor compound, MNK-I1 (Beggs et al., 2015) was investigated. MNK-I1 is structurally distinct from CGP57380 and cercosporamide and is much more specific and does not inhibit the additional kinases affected by CGP57380 (Beggs et al., 2015). It is also much more potent and strongly inhibits MNK activity in 3T3-L1 cells at much lower concentrations (3 μM; see FIG. 14A, 14B; cf. for CGP57380, 50 μM was required to see a similar degree of inhibition of p-eIF4E (see FIG. 3c)). Importantly, given that the cells have to be incubated for at least three days to undergo differentiation, the effect of MNK-I1 on P-eIF4E persists for at least this period of time.

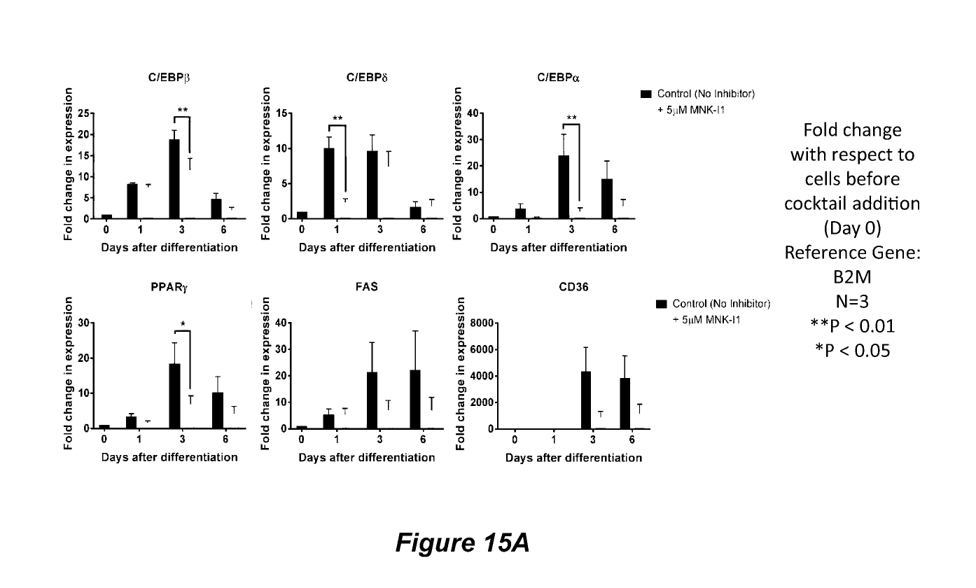

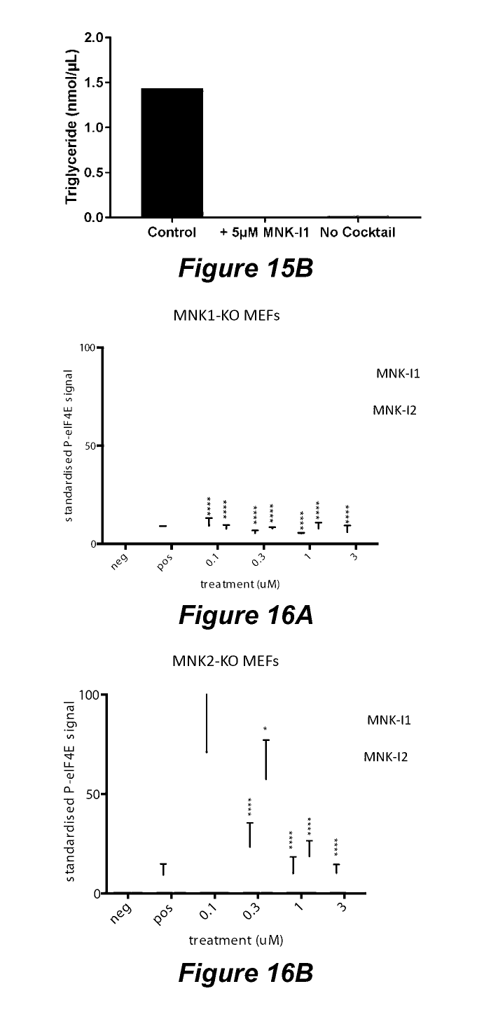

It was found that MNK-I1 impaired the induction of all of the studied adipogenic genes, including the transcription factors Cebpα, Cebpβ, Cebpδ and Pparγ as well as fatty acid synthase and acetyl-CoA carboxylase (Fas and Acc; key enzymes of lipogenesis) and the lipid transporter Cd36 (see FIG. 15A). It also blocked lipid accumulation, as judged by Oil Red O staining (dat not shown) or direct triglyceride assay (FIG. 15B). The fact that the effect of MNK-I1 is not complete (ie not total inhibition) matches the observation that adipocyte number is reduced in MNK2-KO mice, but not completely ablated. This also matches the important finding that MNK-2 KO mice show similar overall fat mass on control diet, although their fat gain is strongly decreased when fed the HFD. This has important clinical implications, since it means patients' weight gain will be reduced or reversed, but that this will not lead to extreme loss of fat tissue.

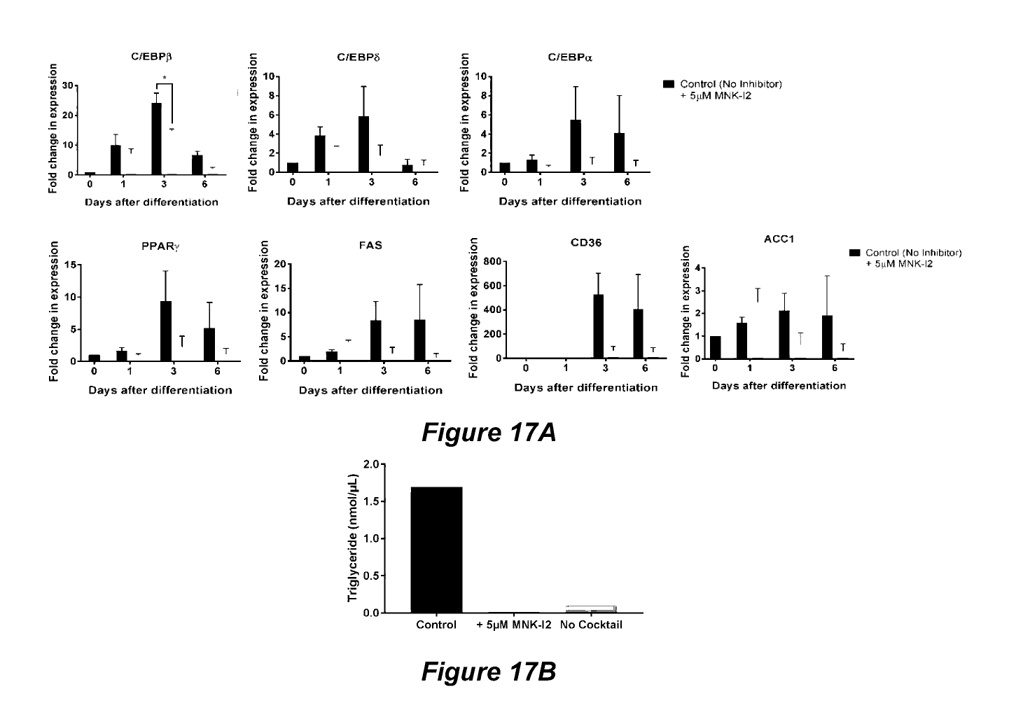

In further studies, a series of compounds similar to MNK-I1 were generated and tested for their ability to inhibit MNKs in 3T3-L1 cells. The structures of the compounds are shown in Table 6 below. Since eIF4E is only phosphorylated by the MNKs, its phosphorylation status is a reliable read-out of intracellular MNK activity. Several of the new compounds inhibited eIF4E phosphorylation in these cells, showing that they are active against MNKs (Table 6). In particular, MNK-12 inhibits P-eIF4E strongly in 3T3-L1 cells (see FIG. 14A) indicating that it is a potent MNK inhibitor. Moreover, to evaluate the relative effect of MNK-I1 and MNK-I2 on MNK1 vs. MNK2, use was made of mouse embryonic fibroblasts (MEFs) from MNK1-KO or MNK2-KO animals. In MNK1-KO MEFs, MNK-I2 inhibited P-eIF4E levels almost completely at all concentrations tested (lowest being 0.1 μM). This is similar to the effect of MNK-I1 (FIG. 16A). In contrast, in MNK2-KO cells, where eIF4E phosphorylation is dependent upon MNK1, MNK-I2 had a weaker inhibitory effect than MNK-I1, indicating it has lower activity against MNK1 than MNK-I1 does (FIG. 16B). These results therefore show selectivity of the MNK-I2 inhibitor for MNK2 over MNK1. MNK2 is the main MNK in 3T3-L1 cells and in adipose tissue (Moore et al., 2016). In additional studies, tests were conducted to assess the effect of the MNK-I2 inhibitor on the induction of differentiation markers in 3T3-L1 cells. It was found that MNK-I2 impaired the induction of all of the genes in the adipogenic programme that was studied (FIG. 17A). The magnitude of the effects of MNK-I2 was similar or, in some cases, greater than that of MNK-I1 (compare FIGS. 15A and 17A). These results are significant in that they show that MNK2-selective inhibitors, like the MNK1+MNK2 inhibitors CGP57380 and MNK-I1, also impair adipogenesis and support the conclusion that MNK2 is the crucial MNK2 isoform in adipose tissue.

Therefore, based on the data obtained from the KO mouse studies (see Example 2) and the experimentation conducted in vitro with 3T3-L1 cells (see Example 3), MNK-2-selective inhibitors offer considerable promise and are likely to be more beneficial in the treatment of pre-diabetic subjects than inhibitors of MNK1 and MNK2.

TABLE 6

| | | Mol. | |
|---|---|---|---|
| Compound | Structure of compound | wt | Comments |
| MNK-7I | 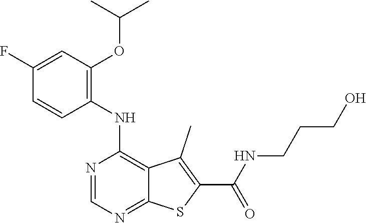 [SMILES: Cc1c(C(=O)NCCCO)sc2ncnc(Nc3ccc(F)cc3OC(C)C)c12] | 418.15 | Partially active |
| MNK-7F | 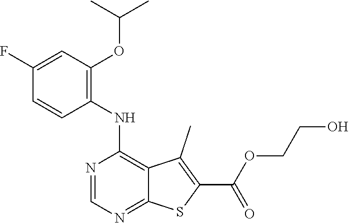 [SMILES: Cc1c(C(=O)OCCO)sc2ncnc(Nc3ccc(F)cc3OC(C)C)c12] | 405.12 | Weak activity |
| MNK-7J | 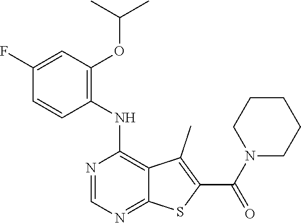 [SMILES: Cc1c(C(=O)N2CCCCC2)sc2ncnc(Nc3ccc(F)cc3OC(C)C)c12] | 428.17 | inactive |
| MNK-7K | 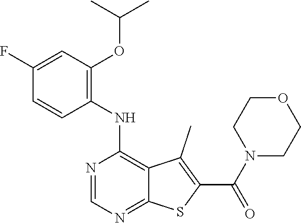 [SMILES: Cc1c(C(=O)N2CCOCC2)sc2ncnc(Nc3ccc(F)cc3OC(C)C)c12] | 430.15 | Almost inactive |
| MNK-7L | 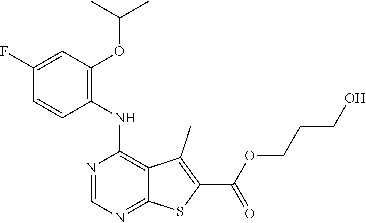 [SMILES: Cc1c(C(=O)OCCCO)sc2ncnc(Nc3ccc(F)cc3OC(C)C)c12] | 419.13 | Partial activity |
| MNK-I2 | 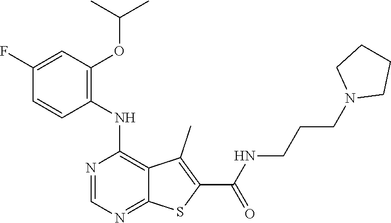 [SMILES: Cc1c(C(=O)NCCCN2CCCC2)sc2ncnc(Nc3ccc(F)cc3OC(C)C)c12] | 471.21 | Good activity, especially against MNK2 |
| MNK-7O | 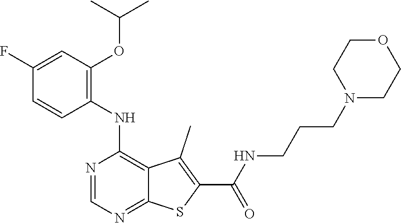 [SMILES: Cc1c(C(=O)NCCCN2CCOCC2)sc2ncnc(Nc3ccc(F)cc3OC(C)C)c12] | 487.21 | Good activity |
| MNK-7P | 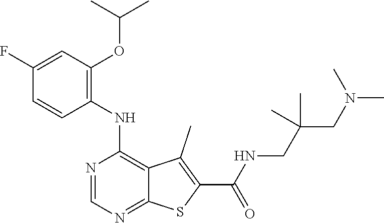 [SMILES: Cc1c(C(=O)NCC(C)(C)CN(C)C)sc2ncnc(Nc3ccc(F)cc3OC(C)C)c12] | 473.23 | Good activity |
| JX-MNK-7S | 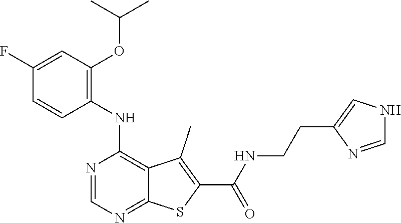 [SMILES: Cc1c(C(=O)NCCc2c[nH]cn2)sc2ncnc(Nc3ccc(F)cc3OC(C)C)c12] | 454.16 | Good activity |
| MNK-7T | 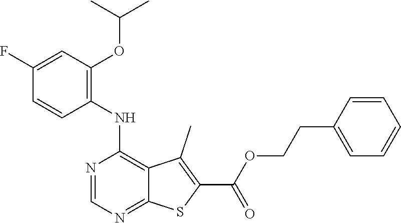 [SMILES: Cc1c(C(=O)OCCc2ccccc2)sc2ncnc(Nc3ccc(F)cc3OC(C)C)c12] | 465.15 | inactive |
| MNK-7U | 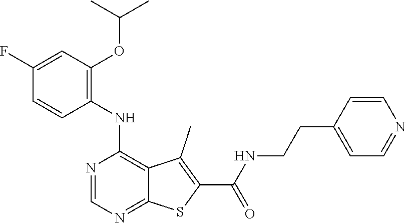 [SMILES: Cc1c(C(=O)NCCc2ccncc2)sc2ncnc(Nc3ccc(F)cc3OC(C)C)c12] | 465.16 | Good activity |
| MNK-7V | 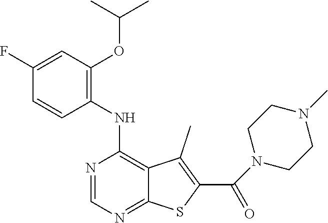 [SMILES: Cc1c(C(=O)N2CCN(C)CC2)sc2ncnc(Nc3ccc(F)cc3OC(C)C)c12] | 443.18 | Partial activity |
| MNK-7W | 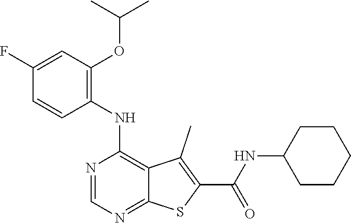 [SMILES: Cc1c(C(=O)NC2CCCCC2)sc2ncnc(Nc3ccc(F)cc3OC(C)C)c12] | 442.18 | Partial activity |
| MNK-7X | 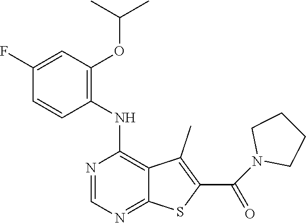 [SMILES: Cc1c(C(=O)N2CCCC2)sc2ncnc(Nc3ccc(F)cc3OC(C)C)c12] | 414.15 | Partial actvitiy |
| MNK-5F | 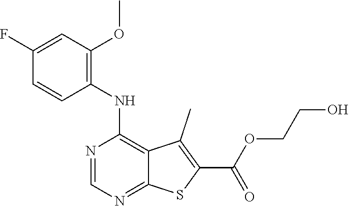 [SMILES: COc1cc(F)ccc1Nc1ncnc2sc(C(=O)OCCO)c(C)c12] | 377.08 | Partial activity |
| MNK-5F | 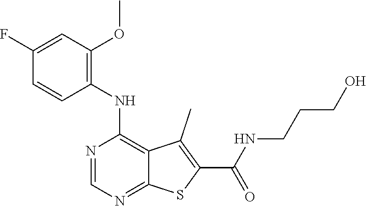 [SMILES: COc1cc(F)ccc1Nc1ncnc2sc(C(=O)NCCCO)c(C)c12] | 390.12 | Good activity |
| MNK-5J | 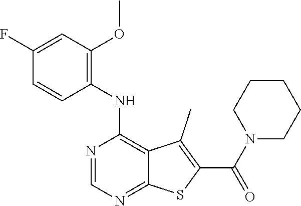 [SMILES: COc1cc(F)ccc1Nc1ncnc2sc(C(=O)N3CCCCC3)c(C)c12] | 400.14 | inactive |
| MNK-5K | 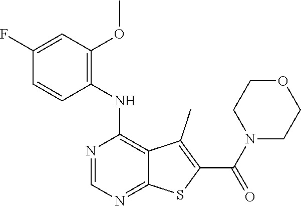 [SMILES: COc1cc(F)ccc1Nc1ncnc2sc(C(=O)N3CCOCC3)c(C)c12] | 402.12 | Almost inactive |
| MNK-5M | 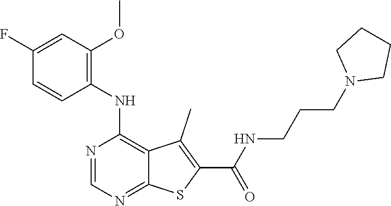 [SMILES: COc1cc(F)ccc1Nc1ncnc2sc(C(=O)NCCCN3CCCC3)c(C)c12] | 443.18 | Good activity |
| MNK-5O | 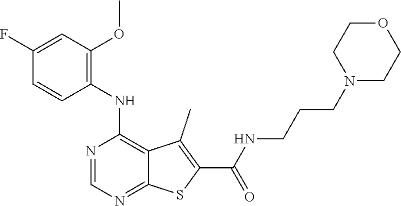 [SMILES: COc1cc(F)ccc1Nc1ncnc2sc(C(=O)NCCCN3CCOCC3)c(C)c12] | 459.17 | Good activity |
| MNK-5P | 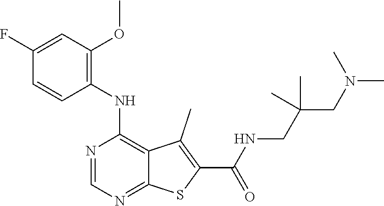 [SMILES: COc1cc(F)ccc1Nc1ncnc2sc(C(=O)NCC(C)(C)CN(C)C)c(C)c12] | 445.19 | Moderate activity |
| MNK-5S | 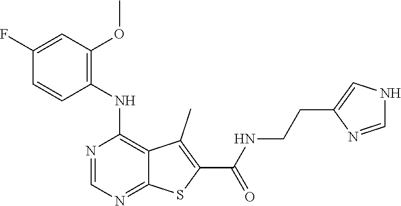 [SMILES: COc1cc(F)ccc1Nc1ncnc2sc(C(=O)NCCc3c[nH]cn3)c(C)c12] | 426.13 | Moderate activity |
| MNK-5U | 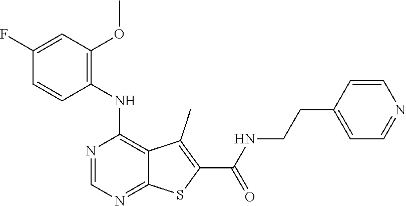 [SMILES: COc1cc(F)ccc1Nc1ncnc2sc(C(=O)NCCc3ccncc3)c(C)c12] | 437.13 | Moderate activity |
| MNK-5V | 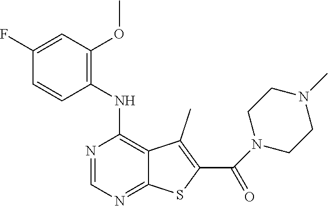 [SMILES: COc1cc(F)ccc1Nc1ncnc2sc(C(=O)N3CCN(C)CC3)c(C)c12] | 415.15 | Almost inactive |
| MNK-5W | 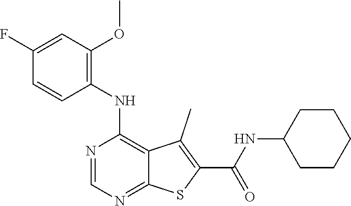 [SMILES: COc1cc(F)ccc1Nc1ncnc2sc(C(=O)NC3CCCCC3)c(C)c12] | 414.15 | Moderate activity |
| MNK-5X | 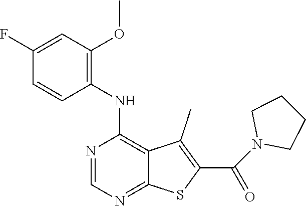 [SMILES: COc1cc(F)ccc1Nc1ncnc2sc(C(=O)N3CCCC3)c(C)c12] | 386.12 | Almost inactive |

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

### REFERENCES

Bain J et al., Biochem J 408(3):297-315 (2007).
Beggs, J E et al., Biochem J 467(1):63-76 (2015).
Benson D A et al., Nucleic Acids Res 28(1):15-18 (2000).
Buxade, M et al., Front Biosci 13:5359-5373 (2008).
Chrestensen, C A et al., J Biol Chem 282(7):4243-4252 (2007).
Hariri, N and L Thibault, Nutr Res Rev 23:270-299 (2010).
Hou, J et al., Oncotarget 3(2): 118-131 (2012).
Knauf, U et al., Mol Cell Biol 21(16):5500-5511 (2001).
Konicek, B W et al., Cancer Res 71(5):1849-1857 (2011).
Liu, R et al., Nucleic Acids Res 42:5083-5096 (2014).
Liu, Y and N S Gray, Nat Chem Biol 2(7):358-364 (2006).
Matthews, D R et al., Diabetologia 28:412-419 (1985).
Moore, C E J et al., Sci Report 6:23476 doi: 10.1038/srep23476 (2016).
Oyarzabal et al., J Med Chem 53:6618 (2010).
Panchal, S K and L Brown, J Biomed Biotechnol 35:1982 (2011).
Rowlett R M et al., Am J Physiol Gastrointest Liver Physiol 294(2):G452-459 (2008).
Scheper, G C et al., Mol Cell Biol 21:743-754 (2001).
Smith, P J et al., J Biol Chem 263:9402-9408 (1988).
Sussman A et al., Eukaryotic cell 3(4):932-943 (2004).
Tschopp, C et al., Mol Cell Biol Res Commun 3:205-211 (2000).
Ueda, T et al., Mol Cell Biol 24:6539-6549 (2004).
Waskiewicz, A J et al., EMBO J 16:1909-1920 (1997).
Wang, X et al., J Biol Chem 273:9373-9377 (1998).
Weischenfeldt, J and B Porse, CSH Protoc 3(12); pdb.prot5080(2008)
Worch, J et al., Oncogene 23(57):9162-9172 (2004).

1.-29. (canceled)

30. A method of treating a subject with impaired fasting glucose pre-diabetes to prevent and/or delay progression of the pre-diabetes to type 2 diabetes, said subject characterised by having a fasting plasma glucose level from 5.5 mmol/l to 6.9 mmol/l, wherein said method comprises administering to the subject a therapeutically effective amount of at least one mitogen-activated protein kinase-interacting kinase (MNK) inhibitor, wherein said MNK inhibitor reduces the biological activity of MNK2 and, optionally, MNK1.

31. The method of claim 30, wherein the MNK inhibitor reduces the biological activity of MNK2.

32. The method of claim 30, wherein the MNK inhibitor shows selectivity to MNK2.

33. The method of claim 30, wherein the MNK inhibitor reduces the biological activity of MNK1.

34. The method of claim 30, wherein the MNK inhibitor reduces the biological activity of MNK1 and MNK2.

35. The method of claim 30, wherein the MNK inhibitor is selected from the group consisting of a small organic molecule, a peptide inhibitor, an inhibitory antibody or fragment thereof, interfering nucleotide molecule, or an aptamer.

36. The method of claim 30, wherein the MNK inhibitor is an ATP competitor.

37. The method of claim 30, wherein the MNK inhibitor is selected from N3-(4-Fluorophenyl)-1 H-pyrazolo-[3,4-d]pyrimidine-3,4-diamine; (9aS)-8-Acetyl-9,9a-dihydro-1,3,7-trihydroxy-9a-methyl-9-oxo-4-dibenzofurancaboxamide, 4-[5-(4-Piperidinyl)-1H-pyrazol-3-yl]pyridine dihydrochloride, 4-(2-(2-fluoropropoxy)-4-fluorophenylamino)-N-(3-(dimethylamino)propyl)-5-methylthieno[2,3-d]pyrimidine-6-carboxamide and 4-(2-isopropoxy)-4-fluorophenylamino)-N-(3-(pyrrolidin-1-yl)propyl)-5-methylthieno[2,3-d]pyrimidine-6-carboxamide.

38. The method of claim 30, wherein the MNK inhibitor is formulated as a pharmaceutical composition optionally in combination with a pharmaceutically or veterinary-acceptable filler, carrier, diluent and/or excipient.

39. The method of claim 30, wherein the method prevents progression of pre-diabetes at the impaired fasting glucose (IFG) stage to the impaired glucose tolerance (IGT) stage.

40. The method of claim 30, wherein the subject is characterised by a fasting plasma glucose level from 6.1 mmol/l to 6.9 mmol/l.
